(19) United States
(12) Patent Application Publication
(10) Pub. No.: US 2023/0416358 A1
(43) Pub. Date: Dec. 28, 2023

(54) COMPOSITIONS AND METHODS FOR TREATING, AMELIORATING, AND/OR PREVENTING DISEASES OR DISORDERS CAUSED BY OR ASSOCIATED WITH DNASE1 AND/OR DNASE1L3 DEFICIENCY

(71) Applicant: YALE UNIVERSITY, New Haven, CT (US)

(72) Inventors: Paul Stabach, New Haven, CT (US); Demetrios Braddock, New Haven, CT (US)

(21) Appl. No.: 18/354,428

(22) Filed: Jul. 18, 2023

Related U.S. Application Data
(63) Continuation of application No. 17/792,101, filed on Jul. 11, 2022.
(60) Provisional application No. 62/959,932, filed on Jan. 11, 2020.

(51) Int. Cl. C07K 16/24; C07K 2/00; C07K 14/00; C12N 9/22

(52) U.S. Cl. CPC C07K 16/247; C12N 9/22; C07K 14/00; C07K 2/00

(57) ABSTRACT

The present disclosure includes novel DNAse1 and/or DNAse1L3 constructs with improved in vivo half-lives, enzymatic stability, and/or developability properties.

### CROSS REFERENCE TO RELATED APPLICATIONS

This application is a continuation of, and claims priority to, U.S. application Ser. No. 17/792,101 filed Jul. 11, 2022, which is a 35 U.S.C. § 371 national phase application of, and claims priority to, International Application No. PCT/US2021/012990 filed Jan. 11, 2021, which claims priority under 35 U.S.C. § 119(e) of U.S. Provisional Patent Application No. 62/959,932 filed Jan. 11, 2020, all of which are hereby incorporated by reference in their entireties.

### REFERENCE TO SEQUENCE LISTING

The Sequence Listing submitted herewith as a text file named “047162-7271US2_Sequence_listing.txt,” created Jul. 10, 2023 and having a size of 66 Kbytes, is herein incorporated by reference pursuant to 37 C.F.R. § 152(e)(5).

### BACKGROUND OF THE DISCLOSURE

Lupus erythematosus (or lupus) is a general term for a group of autoimmune diseases in which the human immune system becomes hyperactive and attacks healthy tissues. Symptoms of these diseases can affect many different body systems, including joints, skin, kidneys, blood cells, heart, and lungs. The most common and most severe form of lupus is systemic lupus erythematosus (SLE).

Lupus symptoms vary from person to person, and may occur sporadically. Common symptoms are joint pain and swelling, and arthritis, mostly in the fingers, hands, wrists, and knees. Other common symptoms include: chest pain during respiration; oral ulcer; fatigue; weight loss; fever with no other cause; general discomfort, uneasiness, or ill feeling (malaise): hair loss; sensitivity to sunlight; a “butterfly” facial rash, seen in about half people with SLE; and swollen lymph nodes. Lupus is a high morbidity condition, with the majority of patients experiencing life-threatening nephritis.

Lupus is thought to be influenced by multiple genes and gene polymorphisms, more than 30 of which have now been linked with the disorder. SLE pathogenesis is related to a reduced ability to clear DNA released from apoptotic cells, the accumulation of which over time triggers an autoimmune response and the formation of the anti-DNA autoantibodies. There are two key features associated with SLE: dysregulated activation of both T and B lymphocytes and the development of anti-DNA autoantibodies-particularly against double-stranded DNA—that are involved in tissue damage. Major histocompatibility complex (MHC) class II genes, certain class II genes [complement components 2 (C2) and 4 (C4A), tumor necrosis factor (TNF) and heat shock protein 70 kD (HSPA1A) alleles] and other non-MHC genes [receptors for Fc fragment of IgG, low affinity IIa and IIIa (FCGR2A and FCGR3A), interleukins 6 and 10 (IL6 and IL10) and B-cell CLL/lymphoma 2 (BCL2)] can each contribute to susceptibility. However, the principal loci and alleles responsible for disease susceptibility are unknown at this time.

Recently a rare autosomal recessive form of SLE with a null mutation in the DNAse1L3 gene was described (Al-Mayouf, et al., 2011, Nature Genetics 43(12), 1186-1188). The DNAse1L3-related SLE was pediatric in onset and correlated with a high frequency of lupus nephritis. Indeed, DNAse1L3-deficient mice were found to rapidly develop antibodies to double-stranded DNA and chromatin, followed later by immune activation, IgG deposition in the kidney glomeruli, and glomerulonephritis. DNAse1L3 is one of three human homologs of DNase I; this enzyme functions as an endonuclease capable of cleaving both single- and double-stranded DNA, is not inhibited by actin, and mediates the breakdown of DNA during apoptosis. On the other hand, DNAse1 binds actin monomers with very high (sub-nanomolar) affinity and actin polymers with lower affinity; actin-bound DNase I is enzymatically inactive. DNAse1 cleaves non-complexed DNA, while DNAse1L3 cleaves chromatin.

Unfortunately, stable and bioavailable DNAse1 and/or DNAse1L3 enzyme biologics designed to treat lupus associated with DNASE1 and/or DNAse1L3 deficiency, as well as other pathologies associated with DNAse1 and/or DNAse1L3 deficiency have not been described to date in the literature.

There is thus a need in the art for compositions and methods that can be used to treat diseases or disorders that are caused by and/or associated with DNAse1 and/or DNAse1L3 deficiency, such as but not limited to SLE. The present disclosure fulfills this need.

### BRIEF SUMMARY OF THE DISCLOSURE

The present disclosure provides certain constructs, such as but not limited to DNAse1-X1-LINKER-Fc-X2 and DNAse1L3-X1-LINKER-Fc-X2, wherein DNAse1, DNAse1L3, X1, X2, LINKER, and Fc are defined elsewhere herein. Further, the disclosure provides certain homodimeric constructs comprising at least one construct of the disclosure.

The present disclosure provides methods of treating, ameliorating, and/or preventing forms of lupus associated with DNAse1 and/or DNAse1L3 deficiency in a subject using certain constructs of the disclosure. Further, the present disclosure provides methods of treating, ameliorating, and/or preventing diseases and/or disorders associated with inefficient NET hydrolysis (NETolysis) in a subject using certain constructs of the disclosure. Further, the present disclosure provides methods of treating, ameliorating, and/or preventing an autoimmune disorder associated with DNAse1 and/or DNAse1L3 deficiency in a subject using certain constructs of the disclosure. Further, the present disclosure provides methods of treating, ameliorating, and/or preventing pathologic thrombosis in a subject using certain constructs of the disclosure. Further, the present disclosure provides methods of treating, ameliorating, and/or preventing a myocardial infarction in a subject using certain constructs of the disclosure.

Further, the present disclosure provides methods of treating, ameliorating, and/or preventing cancer metastasis in a subject using certain constructs of the disclosure.

### BRIEF DESCRIPTION OF THE DRAWINGS

The following detailed description of illustrative embodiments of the disclosure will be better understood when read in conjunction with the appended drawings. For the purpose of illustrating the disclosure, exemplary embodiments are shown in the drawings. It should be understood, however, that the disclosure is not limited to the precise arrangements and instrumentalities of the embodiments shown in the drawings.

FIG. 1 illustrates neutrophil extracellular trap (NET) formation. Scanning electron microscopy of neutrophil (marked as A) casting a net (marked as B) entrapping Helicobacter pylori bacteria (some of which are marked as C). Image taken from Kumamoto T, et al., 2006, Eur Heart J. 27(17):2081-7.

FIG. 2 illustrates a non-limiting DNAse1-Fc construct of the disclosure, with certain contemplated point mutations highlighted.

FIG. 3 illustrates a non-limiting DNAse1L3-Fc construct of the disclosure, with certain contemplated point mutations highlighted.

FIG. 4 illustrates a non-limiting DNAse1-Fc construct of the disclosure, with certain contemplated point mutations highlighted.

FIG. 5 illustrates non-limiting constructs of the disclosure, with certain contemplated point mutations highlighted. In certain embodiments, certain mutations render the rDNAse hyperactive and/or render the rDNAse actin-resistant (i.e., has decreased affinity for actin) and/or increase the construct's half-life. The non-limiting alignment of amino acid sequences of mouse DNAse1 (SEQ ID NO:42) and mouse DNAse1L3 (SEQ ID NO:43) is illustrated.

FIG. 6 illustrates non-limiting constructs of the disclosure, with certain contemplated point mutations highlighted. In certain embodiments, the construct lacks at least a portion of the DNAse1L3 nuclear localization domain.

FIG. 7 illustrates a gel indicating that certain DNAse1L3 clones cleave chromatin, but that is not the case for certain DNAse1 clones.

FIG. 8 illustrates a non-limiting construct of the disclosure. In certain embodiments, the DNAse1 polypeptide is fused with the C-terminus tail of DNAse1L3.

FIG. 9 illustrates certain aspects of production and purification of DNAse-Fc constructs.

FIGS. 10A-10B illustrate in vivo pharmacodynamics of certain NET degrading constructs of the disclosure.

FIG. 11 illustrates a non-limiting purification gel of certain NET degrading constructs of the disclosure.

FIG. 12 illustrates the finding that a non-limiting optimized heterodimer comprising DNAse1 and DNAse1L3 demonstrates increased NET degradation compared to optimized DNAse1 or DNAse1L3 constructs alone. The DNAse degrading activity of various constructs described in the application are shown against DNA alone (Top gel) and protein associated DNA (bottom gel). A purified heterodimer comprised of DNAse1 and DNAse1L3 (heterodimer of constructs 1669 and 1689) is shown in lanes 2 and 5, various purified optimized constructs of DNAse1 are shown in lanes 1,3,4 and 6, and a purified, optimized DNAse1L3 construct is in lane 7. As shown in the figure, only the heterodimer digests both the plasmid DNA (upper gel) and the Chromatin DNA (lower gel), as identified by the reduced size of the DNA bands in the gel.

### DETAILED DESCRIPTION OF THE DISCLOSURE

The present disclosure relates, in one aspect, to the discovery that certain constructs can be used to treat, ameliorate, and/or prevent diseases or disorders associated with DNAse1 and/or DNAse1L3 deficiency.

In certain embodiments, the constructs contemplated herein can be used to treat, ameliorate, and/or prevent forms of lupus (including SLE) associated with DNAse1L3 deficiency.

In certain embodiments, the constructs contemplated herein can be used to treat, ameliorate, and/or prevent diseases and/or disorders associated with inefficient NET hydrolysis (“NETolysis”).

In certain embodiments, the constructs contemplated herein can be used to treat, ameliorate, and/or prevent autoimmune disorders such as but not limited to lupus (including SLE), thyroid autoimmune disease, and Hypocomplementeric Urticarial Vasculitis Syndrome (HUVS).

In certain embodiments, the constructs contemplated herein can be used to treat, ameliorate, and/or prevent pathologic thrombosis, such as but not limited to microvascular thrombosis, venous thrombosis, and/or arterial thrombosis. In certain embodiments, the pathologic thrombosis comprises neutrophilic thrombosis, which includes but is not limited to Anti-Neutrophilic Cytoplasmic Autoantibodies (ANCA) vasculitis, Thrombotic thrombocytopenic purpura (TTP), and Bechet's (or Behcet's) disease or syndrome. In certain embodiments, the pathologic thrombosis comprises thrombosis leading to strokes.

In certain embodiments, the constructs contemplated herein can be used to treat, ameliorate, and/or prevent myocardial infarctions.

In certain embodiments, the constructs contemplated herein can be used to treat, ameliorate, and/or prevent spread and progression of cancer (e.g., cancer metastasis).

The present disclosure provides stable and bioavailable constructs comprising DNAse1L3 and/or DNAse1 polypeptides (or fragments, rearrangements, (point) mutations, truncations, and/or any other modifications and/or analogues and/or derivatives thereof) fused with certain proteins. In certain embodiments, the constructs contemplated herein have increased bioavailability and/or developability over the DNAse1 L3 and/or DNAse1 polypeptides known in the art. In certain embodiments, the constructs contemplated herein have enhanced enzymatic activity over the DNAse1L3 and/or DNAse1 polypeptides known in the art. In certain embodiments, the constructs contemplated herein have improved pharmacokinetic behavior over the DNAse1L3 and/or DNAse1 polypeptides known in the art. In certain embodiments, the constructs contemplated herein have enhanced stability over the DNAse1L3 and/or DNAse1 polypeptides known in the art.

In certain embodiments, the in vivo half-life of a construct of the disclosure is at least about 1.5, 2, 2.5, 3, 4, 5, 6, 7, 8, 9, 10, 12, 14, 16, 18, and/or 20 times higher than the DNAse1 and/or DNAse1L3 polypeptides described in the art. In other embodiments, the constructs of the disclosure are administered to the subject at a lower dose and/or at a lower frequency than other DNAse1 and/or DNAse1L3 polypeptides in the art. In yet other embodiments, the constructs of the disclosure are administered to the subject once a month, twice a month, three times a month, and/or four times a month. In yet other embodiments, the lower frequency administration of the constructs of the disclosure results in better patient compliance and/or increased efficacy as compared with other DNAse1 and/or DNAse1L3 polypeptides in the art.

The constructs of the disclosure can be used to treat diseases or disorders that are caused by and/or associated with and/or related to decreased and/or inefficient and/or suboptimal neutrophil extracellular traps (NETs) degradation and/or clearance and/or hydrolysis. Polymorphonuclear leukocytes (PMNs), the most abundant form of white blood cells, circulate in tissues and blood, where they seek out invading micro-organisms. If invading micro-organisms are encountered by PMN's, the cells respond with an array of mechanisms to combat the infection, including phagocytosis, the release of stored antimicrobial compounds in a process called degranulation, and, as a last resort, a self-destructive process wherein the PMN “explodes,” releasing a web of entrapping DNA and cytotoxic material known as “neutrophilic extracellular traps” or NETs. NETs are extracellular, neutrophil-derived DNA webs that trap invading pathogens. The backbone of NETs is a sticky chromatin web attached to which are an assortment of antimicrobial cytotoxic proteins and peptides that are released along with the chromatin when PMN degranulate in response to infectious stimuli. The high concentration of antimicrobial compounds maintained by NETs in close proximity to invading organisms increases the potency of the cytotoxic agents, thereby neutralizing the invading pathogens to prevent their spread and eliminate the threat of infection.

Notwithstanding their beneficial role fighting infection, NETs must be cleared quickly and efficiently from tissues and the circulation, and failure to do so has serious pathologic consequences. Specifically, diseases associated with inefficient NET hydrolysis (“NETolysis”) include autoimmune disorders such as lupus, pathologic thrombosis (such as but not limited to thrombosis leading to strokes), and myocardial infarctions, and the spread and progression of cancer.

NETs are typically degraded by blood-based metalloenzymes, and several circulating enzyme isoforms hydrolyze the high energy bonds in DNA to effect NETolysis. To do so, different enzyme isoforms recognize DNA as free nucleic acid or in associated with proteins such as the chromatin in the protein backbone of NETs. Loss of function mutations in these enzymes have been identified in Systemic Lupus Erythematosus (SLE), including hereditable and highly aggressive forms of SLE presenting in the pediatric population. In addition, NETs foster cancer progression and metastasis, and inhibition of NETs has been shown to decrease cancer metastasis in murine models.

NETs in Tumor Progression and Metastasis: Currently, locoregional control of cancer via complete surgical resection constitutes the major curative modality for all forms of solid tumors, and significantly improves the disease free and overall survival of most tumors in the adjuvant setting. Control of postsurgical distant metastasis is thus of paramount importance to patient outcomes, and currently systemic chemotherapy is used to control tumor recurrence with variable results. Infectious complications associated with cancer treatment have long been associated with adverse oncologic outcomes independent of the morbidity associated with the infectious complication, a phenomenon observed across a broad range of malignancies including tumors of the lung, breast, colon, and esophagus. Specifically, post-treatment infection following surgery or chemotherapy is strongly associated with an increased rate of death from metastatic disease. Regrettably infectious complications are a frequent occurrence in oncology, approaching 40% in some series. Recently, the association between infection and tumor recurrence has been linked to the presence of NETs in the tumor microenvironment. Degranulation of neutrophils induced by infection increases NETs, which bind tumor cells in the blood and appear to support their metastatic progression.

After finding that infection induced NET formation may lead to the progression and spread of cancer, investigators examined the ability of cancer cells themselves to induce NET formation and found that highly aggressive cancer cells can induce NET formation in the absence of infection. It appears that cancer cells “highjack” the immune system by inducing neutrophils to extrude NETs in order to hitch a ride through the circulatory system and support their attachment to distant sites of metastasis. In support of these findings, the levels of circulating NETs in cancer patients with advanced esophageal, lung, and GI cancer correlates directly with their cancer stage, i.e., with the presence of the cancer at distant sites from the primary tumor (i.e., stage I-II cancer has less NETs in blood than stage III-IV cancer). The findings suggest that NET number can be used as an independent biomarker for cancer progression in these aggressive forms of cancer. Also supporting the notion that NETs aid the metastasis of aggressive forms of cancer are preclinical murine models of lung, GI, and breast cancer in which the inhibition of NETs prevented adherence the circulating tumor cells to liver sinusoids, and decreased the appearance of lung metastasis. These studies demonstrate that NETs promote liver and lung metastasis in mouse models of human tumors, and that targeting NETs can inhibit the metastatic spread of tumors.

The current studies of the effects of NETs in cancer demonstrate that circulating NET levels are prognostically significant biomarkers for tumor progression and metastasis, that human cancer cells are capable of inducing metastasis-promoting NETs to aid in their growth and spread, and that inhibition of NET formation can suppress tumor progression and metastasis in murine models of human GI, lung and breast cancer. In total, the studies support use of NET based therapies in cancer therapeutics.

Autoimmune disease with focus on Systemic Lupus Erythematosus (SLE): The inadequate clearance of dead and apoptotic cells has long been believed to be directly tied to the pathogenesis of autoimmune disease, including some forms of SLE. This notion stems from the finding, first reported 40 years ago, that patients with SLE have lower serum DNAse1 activity than healthy subjects, which is the enzyme in the blood that is primarily responsible for the removal of DNA from dead and apoptotic cells, and the clearance of NETs. In addition, low DNAse1 activity is associated with the active phase of nephropathy in lupus (type III or type IV), implicating DNAse1 activity with the development of SLE nephropathy.

Responding to these clinical observations, researchers engineered a transgenic DNAse1 knockout mouse lacking DNAse1 activity, and discovered that these mice recapitulated the clinical and biochemical phenotype present in human SLE, including the appearance of double stranded DNA (dsDNA) antibodies, glomerulonephritis resulting from the deposition of autoantibodies on the glomerular surface, and perivascular infiltrates. Strikingly, DNAse1 deficient mice also recapitulated the female bias seen in humans with SLE.

Inactivating mutations in DNAse1 were present two Japanese patients with pediatric lupus in a heterozygous pattern (Yasutomo I et al., 2001, Nat Genet. 2001; 28(4):313-4). Importantly, these girls had very low DNAse1 activity and very high titers of anti-nucleosome and anti-double-stranded DNA (dsDNA) antibodies. Following this study, homozygous inactivating mutations in DNAse1L3 were found in several Arabic families with children who presented with lupus as early as 2 years of age (Al-Mayouf, et al., 2011, Nature Genetics 43(12), 1186-1188), suggesting a very aggressive form of the disease. Additional evidence tying DNAse1 and DNAse1L3 to human autoimmune disease were the identification of loss of function variants of DNase1 in Spanish patients with SLE, in Caucasian patients with thyroid autoimmune disease, and in Turkish and Italian patients with Hypocomplementeric Urticarial Vasculitis Syndrome, an autoimmune disorder with overlapping clinical features of SLE.

While the findings support the notion that DNAse1 and DNAse1L3 are involved in the pathogenesis of human autoimmune disease, subsequent studies determined that the frequency of these mutations are very rare, essentially below 1% in patients with the autoimmune disease. Finally, the identification of a single nucleotide polymorphism (SNP) in a Caucasian specific allele of DNAse1L3 was identified in SLE producing completely inactive enzyme. The allele frequency was found in three Caucasian population—Mexican, Turkish and German—with a predicted minor allele frequency of 0.017, 0.052, and 0.077 respectively. Because homozygous loss of function in DNase1L3 is required to predispose patients to autoimmune disease, the number of genetically defined cases resulting from this form of DNAse1 L3 deficiency is also expected to be very low.

This picture changed dramatically with the identification of a common polymorphism of DNAse1 that was identified in lupus patients. The SNP, which is a Q244R polymorphism in exon 8, also identified as rs1053874, is distributed worldwide with a minor allelic frequency of 0.494. The R244 form of the allele was found to be strongly associated with the production of anti-ribonuclear protein (anti-RNP) antibodies in Korean lupus patients and was significantly associated with SLE susceptibility in Argentinian patients. Following this association, Japanese researchers determined the R244 variant to have half the enzyme activity as the Q244 variant, thereby accounting for the greater propensity of autoimmune disease in patients possessing the R244 polymorphism. The same researchers then went on to systematically evaluated all non-synonymous DNAse1 SNPs in the Ensembl database (ensemble dot org), eventually identifying 60 loss of function variants of DNAse1, all of which are potentially pathogenic. The occurrence of most of these forms are quite rare, but the frequency of the most common variant (rs1053874-Q244R, with an MAR of 0.494) is expected to be present as homozygous in about 25% of the population worldwide.

NETs in Pathologic Thrombosis: Another area of therapeutic intervention in which targeting NETs may prove therapeutic is vascular thrombosis, such as but not limited to microvascular thrombosis, venous thrombosis, and arterial thrombosis. In certain embodiments, the disclosure contemplates neutrophilic thrombosis, which includes but is not limited to Anti-Neutrophilic Cytoplasmic Autoantibodies (ANCA) vasculitis, Thrombotic thrombocytopenic purpura (TTP), and Bechet's (or Behcet's) disease or syndrome. NETs are pro-thrombotic, and may enhance thrombosis in pathologic settings such as cancer and myocardial infarction (Thalin C, et al., 2019, Arterioscler Thromb Vasc Biol. 39(9):1724-38). Serum DNAse1 activity abruptly increases in the early phase of acute myocardial infarction, and the low activity polymorphism of DNAse1 described above (rs1053874) was significantly increased in Japanese patients undergoing active myocardial infarction (as oppose to stable angina). These findings direct implicate reduced DNAse1 function to myocardial infarction.

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein the terms “alteration,” “defect,” “variation” or “mutation” refer to a mutation in a gene in a cell that affects the function, activity, expression (transcription or translation) or conformation of the polypeptide it encodes, including missense and nonsense mutations, insertions, deletions, frameshifts and premature terminations.

The term “antibody,” as used herein, refers to an immunoglobulin molecule that is able to specifically bind to a specific epitope on an antigen. Antibodies can be intact immunoglobulins derived from natural sources or from recombinant sources and can be immunoreactive portions of intact immunoglobulins.

As used herein, the term “AUC” refers to the area under the plasma drug concentration-time curve (AUC) and correlates with actual body exposure to drug after administration of a dose of the drug. In certain embodiments, the AUC is expressed in mg*h/L. The AUC can be used to measure bioavailability of a drug, which is the fraction of unchanged drug that is absorbed intact and reaches the site of action, or the systemic circulation following administration by any route.

AUC can be calculated used Linear Trapezoidal method or Logarithmic Trapezoidal method. The Linear Trapezoidal method uses linear interpolation between data points to calculate the AUC. This method is required by the OGD and FDA, and is the standard for bioequivalence trials. For a given time interval (t₁−t₂), the AUC can be calculated as follows:

$$AUC = \frac{1}{2} ( {C}_{1} + {C}_{2} ) ( {t}_{2} - {t}_{1} )$$

wherein C₁ and C₂ are the average concentration over the time interval (t₁ and t₂).

The Logarithmic Trapezoidal method uses logarithmic interpolation between data points to calculate the AUC. This method is more accurate when concentrations are decreasing because drug elimination is exponential (which makes it linear on a logarithmic scale). For a given time interval (t₁−t₂), the AUC can be calculated as follows (assuming that C₁>C₂):

$$AUC = \frac{{C}_{1} - {C}_{2}}{ln ( {C}_{1} ) - ln ( {C}_{2} )} ( {t}_{2} - {t}_{1} )$$

The term “bioavailability” as used herein refers to the extent and rate at which the active moiety (protein or drug or metabolite) enters systemic circulation, thereby accessing the site of action, or the systemic circulation following administration by any route. Bioavailability of an active moiety is largely determined by the properties of the dosage form, which depend partly on its design and manufacture. Differences in bioavailability among formulations of a given drug or protein can have clinical significance; thus, knowing whether drug formulations are equivalent is essential. The most reliable measure of a drug's or protein's bioavailability is area under the plasma concentration-time curve (AUC). AUC is directly proportional to the total amount of unchanged drug or therapeutic protein that reaches systemic circulation. Drug or therapeutic protein may be considered bioequivalent in extent and rate of absorption if their plasma concentration curves are essentially superimposable. For an intravenous dose of a drug, bioavailability is defined as unity. For drug administered by other routes of administration, bioavailability is often less than unity. Incomplete bioavailability may be due to a number of factors that can be subdivided into categories of dosage form effects, membrane effects, and site of administration effect. Half-life and AUC provide information about the bioavailability of a drug or biologic.

As used herein, the terms “conservative variation” or “conservative substitution” as used herein refers to the replacement of an amino acid residue by another, biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine.

As used herein, a “construct” of the disclosure refers to a fusion polypeptide comprising a DNAse1 and/or DNAse1L3 polypeptide, or any fragments, rearrangements, (point) mutations, truncations, or any other modifications and/or analogues and/or derivatives thereof.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “DNAse1” refers to deoxyribonuclease-1 (UniProtKB=P24855). The sequence of human DNAse1 is provided herein (SEQ ID NO: 1). In certain embodiments, the signal peptide of DNAse1 corresponds to residues 1-22 of SEQ ID NO: 1.

```
SEQ ID NO: 1
                10 20 30 40
MRGMKLLGAL LALAALLQGA VSLKIAAFNI QTFGETKMSN
                50 60 70 80
ATLVSYIVQI LSRYDIALVQ EVRDSHLTAV GKLLDNINQD
                90 100 110 120
APDTYHYVVS EPLGRNSYKE RYLFVYRPDQ VSAVDSYYYD
              130 140 150 160
DGCEPCGNDT FNREPAIVRF ESRFTEVREF AIVPLHAAPG
              170 180 190 200
DAVAEIDALY DVYLDVQEKW GLEDVMLMGD FNAGCSYVRP
              210 220 230 240
SQWSSIRLWT SPTFQWLIPD SADTTATPTH CAYDRIVVAG
              250 260 270 280
MLLRGAVVPD SALPENEQAA YGLSDQLAQA ISDHYPVEVM
LK
The sequence of mouse DNAse1 is provided
herein
(SEQ ID NO: 29):
MRYTGLMGTLLTLVNLLQLAGTLRIAAFNIRTFGETKMSNATLSVYFVKI
LSRYDIAVIQEVRDSHLVAVGKLLDELNRDKPDTYRYVVSEPLGRKSYKE
QYLFVYRPDQVSILDSYQYDDGCECGNDTFSREPAIVKFFSPYTEVQEFA
IVPLHAAPTEAVSEIDALYDVYLDVWOKWGLEDIMEMGDENAGCSYVTSS
QWSSIRLRTSPIFQWLIPDSADTTVTSTHCAYDRIVVAGALLQAAVVPNS
AVPEDFQAEYGLSNQLABAISDHYPVEVTLRKI
```

The sequence alignment of human DNAse1 (SEQ ID NO: 1, sequence ‘1’ below) and mouse DNAse1 (SEQ ID NO-29, sequence ‘2’ below) follows:

| DNAse1 | | |
|---|---|---|
| 1 | MRGMKLLGALLALAALLQGAVSLKIAAFNIQTFGETKMSNATLVSYIVQILSRYDIALVQ | 60 |
| 2 | MRYTGLMGTLLTLVNLLQLAGTLRIAAFNIRTFGETKMSNATLSVYFVKILSRYDIAVIQ | 60 |
| | ** *:*:**:*. *** * :*:******:************ *:*:********::* | |
| 1 | EVRDSHLTAVGKLLDNLNQDAPDTYHYVVSEPLGRNSYKERYLFVYRPDQVSAVDSYYYD | 120 |
| 2 | EVRDSHLVAVGKLLDELNRDKPDTYRYVVSEPLGRKSYKEQYLFVYRPDQVSILDSYQYD | 120 |
| | *******.*******:**:* ****:*********:****:*********** :*** ** | |
| 1 | DGCEPCGNDTENREPAIVRFFSRFTEVREFAIVPLHAAPGDAVAEIDALYDVYLDVQEKW | 180 |
| 2 | DGCE-CGNDTFSREPAIVKFFSPYTEVQEFAIVPLHAAPTEAVSEIDALYDVYLDVWQKW | 179 |
| | **** ******.******:*** :***:*********** :**:************ :** | |
| 1 | GLEDVMLMGDFNAGCSYVRPSQWSSIRLWTSPTFQWLIPDSADTTATPTHCAYDRIVVAG | 240 |
| 2 | GLEDIMFMGDENAGCSYVTSSQWSSIRLRTSPIFQWLIPDSADTTVTSTHCAYDRIVVAG | 239 |
| | ****:*:*********** ******** *** ************.* ************ | |
| 1 | MLLRGAVVPDSALPENFQAAYGLSDQLAQAISDHYPVEVMLK-- | 282 |
| 2 | ALLQAAVVPNSAVPEDEQAEYGLSNQLAEAISDHYPVEVTLRKI | 283 |
| | **:.****:**:**:*** ****:***:********** *: | |

As used herein, “human DNAse1” refers to the human DNAse1 sequence as described herein, or any fragments, rearrangements, (point) mutations, truncations, or any other modifications and/or analogues and/or derivatives thereof. As used herein, the term “enzymatically active” with respect to DNAse1 is defined as being capable of binding and hydrolyzing DNA.

As used herein, the term “DNAse1 L3” refers to deoxyribonuclease gamma (UniProtKB=Q13609). The sequence of human DNAse1 L3 is provided herein (SEQ ID NO:2). In certain embodiments, the signal peptide of DNAse1 L3 corresponds to residues 1-20 of SEQ ID NO:2. In certain embodiments, the nuclear localization signal of DNAse1 L3 corresponds to residues 296-304 of SEQ ID NO:2. In certain embodiments, the nuclear localization signal of DNAse1L3 corresponds to residues 292-304 of SEQ ID NO:2. In certain embodiments, the nuclear localization signal of DNAse1L3 corresponds to residues 291-305 of SEQ ID NO:2. In certain embodiments, the nuclear localization signal of DNAse1 L3 corresponds to residues A-B of SEQ ID NO:2, wherein A ranges from 291 to 296 and B ranges from 304 to 305.

| SEQ ID NO: 2 | | |
|---|---|---|
| 10 20 30 40 50 | | |
| MSRELAPLLL LLLSIHSALA MRICSENVRS FGESKQEDKN AMDVIVKVIK | | |
| 60 70 80 90 100 | | |
| RCDIILVMEI KDSNNRICPI LMEKLNRNSR RGITYNYVIS SRLGRNTYKE | | |
| 110 120 130 140 150 | | |
| QYAFLYKEKL VSVKRSYHYH DYQDGDADVF SREPFVVWEQ SPHTAVKDEV | | |
| 160 170 180 190 200 | | |
| IIPLHTTPET SVKEIDELVE VYTDVKHRWK AENFIFMGDE NAGCSYVPKK | | |
| 210 220 230 240 250 | | |
| AWKNIRLRTD PREVWLIGDQ EDTTVKKSTN CAYDRIVERG QEIVSSVVPK | | |
| 260 270 280 290 300 | | |
| SNSVEDFQKA YKLTEEEALD VSDHEPVEFK LQSSRAFTNS KKSVTLRKKT | | |
| KSKRS | | |
| The sequence of mouse DNAse1L3 is provided herein | | |
| (SEQ ID NO: 30): | | |
| MSLHPASPRLASLLLFILALHDTLALRLCSENVRSEGASKKENHEAMDIIVKIIKRCDLILLMEIKDSSN | | |
| NICPMLMEKLNGNSRRSTTYNYVISSRLGRNTYKEQYAFVYKEKLVSVKTKYHYHDYQDGDTDVESREPE | | |
| VVWFHSPFTAVKDFVIVPLHTTPETSVKEIDELVDVYTDVRSQWKTENFIFMGDENAGCSYVPKKAWQNI | | |
| RLRTDPKFVWLIGDQEDTTVKKSTSCAYDRIVLCGQEIVNSVVPRSSGVEDFQKAYDLSEEEALDVSDHF | | |
| PVEFKLQSSRAFTNNRKSVSLKKRKKGNRS | | |
| The sequence alignment of human DNAse1L3 (SEQ ID | | |
| NO: 2, sequence ‘1’ below) and mouse DNAseIL3 | | |
| (SEQ ID NO: 30, sequence ‘2’ below) follows: | | |
| DNAse1L3 | | |
| 1 | -----MSRELAPLLLLLLSIHSALAMRICSENVRSFGESKQEDKNAMDVIVKVIKRCDII | 55 |
| 2 | MSLHPASPRLASLLLFILALHDTLALRLCSENVRSFGASKKENHEAMDIIVKIIKRCDLI | 60 |
| | * .** ***::*::*.:**:*:********* **:*:::***:***:*****:* | |
| 1 | LVMEIKDSNNRICPILMEKLNRNSRRGITYNYVISSRLGRKTYKEQYAFLYKEKLVSVKR | 115 |
| 2 | LLMEIKDSSNNICPMIMEKLNGNSRRSTTYNYVISSRLGRNTYKEQYAFVYKEKLVSVKT | 120 |
| | *:******.*.***:****** ****. *********.**:********:********* | |
| 1 | SYHYHDYQDGDADVESREPFVVWFQSPHTAVKDFVIIPLHTTPETSVKEIDELVEVYTDV | 175 |
| 2 | KYHYHDYQDGDTDVFSREPFVVWFHSPFTAVKDFVIVPLHTTPETSVKEIDELVDVYTDV | 180 |
| | .**********:************:**.********:*****************:***** | |
| 1 | KHRWKAENFIFMGDENAGCSYVPKKAWKNIRLRTDPRFVWLIGDQEDTTVKKSTNCAYDR | 235 |
| 2 | RSQWKTENFIFMGDFNAGCSYVPKKAWQNIRLRTDPKFVWLIGDQEDTTVKKSTSCAYDR | 240 |
| | : :**:*********************:********:*****************.***** | |
| 1 | IVLRGQEIVSSVVPKSNSVFDFQKAYKLTEEEALDVSDHFPVEFKLQSSRAFTNSKKSVT | 295 |
| 2 | IVLCGQEIVNSVVPRSSGVFDFQKAYDLSEEEALDVSDHFPVEFKLQSSRAFTNNRKSVS | 300 |
| | *** *****.****:*..********.*:*************************.:***: | |
| 1 | LRKKTKSKRS | 305 |
| 2 | LKKRKKGNRS | 310 |
| | *:*:.*.:** | |

As used herein, “human DNAse1L3” refers to the human DNAse1L3 sequence as described herein, or any fragments, rearrangements, (point) mutations, truncations, or any other modifications and/or analogues and/or derivatives thereof. As used herein, the term “enzymatically active” with respect to DNAse1L3 is defined as being capable of binding and hydrolyzing DNA.

As used herein, the term “DNAse1-Fc” refers to a DNAse1 polypeptide recombinantly fused and/or chemically conjugated (including both covalent and non-covalent conjugations) to an FcR binding domain of an IgG molecule (preferably, a human IgG). In certain embodiments, the C-terminus of DNAse1 is fused or conjugated to the N-terminus of the FcR binding domain. In certain embodiments, the N-terminus of DNAse1 is fused or conjugated to the C-terminus of the FcR binding domain.

As used herein, the term “DNAse1L3-Fc” refers to a DNAse1L3 polypeptide recombinantly fused and/or chemically conjugated (including both covalent and non-covalent conjugations) to an FcR binding domain of an IgG molecule (preferably, a human IgG). In certain embodiments, the C-terminus of DNAse1L3 is fused or conjugated to the N-terminus of the FcR binding domain. In certain embodiments, the N-terminus of DNAse1L3 is fused or conjugated to the C-terminus of the FcR binding domain.

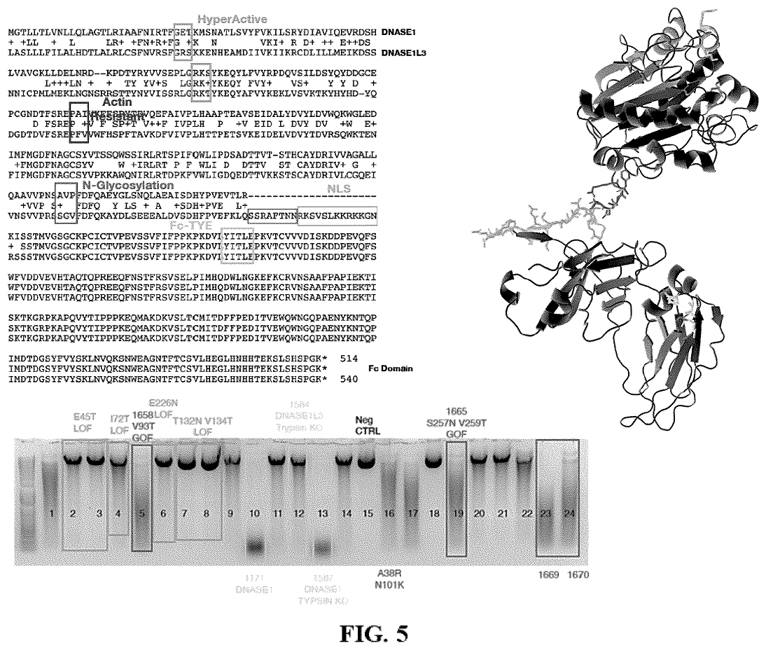

The sequence alignment of mouse DNAse1 (SEQ ID NO:42, ‘query’ below) and mouse DNAse1 L3 (SEQ ID NO:43, ‘Sbjct’ below), as shown in FIG. 5 herein, follows:

| | | | |
|---|---|---|---|
| Query | 7 | MGTLLTLVNLLQLAGTLRIAAFNIRTFGETKMSNATLSVYFVKILSRYDIAVIQEVRDSH | 66 |
| | | + +LL + L LR+ +FN+R+FG +K N VKI+ R D+ ++ E++DS | |
| Sbjct | 10 | LASLLLFILALHDTLALRLCSFNVRSFGPSKKENHEAMDIIVKIIKRCDLILLMEIKDSS | 69 |
| Query | 67 | LVAVGKLLDELNRD--KPDTYRYVVSEPLGRKSYKEQYLEVYRPDQVSILDSYQYDDGCE | 124 |
| | | L+++LN + + TY YV+S LGRK+YKEQY FVY+ VS+ Y Y D + | |
| Sbjct | 70 | NNICPMLMEKLNGNSRRSTTYNYVISSRLGRKTYKEQYAFVYKEKLVSVKTKYHYHD-YQ | 128 |
| Query | 125 | PCGNDTFSREPAIVKFFSPYTEVQEFAIVPLHAAPTEAVSEIDALYDVYLDVWQKWGLED | 184 |
| | | D FSREP +V F SP+T V++F IVPLH P +V EID L DVY DV +W E+ | |
| Sbjct | 129 | DGDTDVESREPFVVWFHSPFTAVKDFVIVPLHTTPETSVKEIDELVDVYTDVRSQWKTEN | 188 |
| Query | 185 | IMFMGDENAGCSYVTSSQWSSIRLRTSPIFQWLIPDSADTTVT-STHCAYDRIVVAGALL | 243 |
| | | +FMGDFNAGCSYV W +IRLRT P F WLI D DTTV ST CAYDRIV+ G + | |
| Sbjct | 189 | FIFMGDENAGCSYVPKKAWQNIRLRTDPKFVWLIGDQEDTTVKKSTSCAYDRIVLCGQEI | 248 |
| Query | 244 | QAAVVPNSAVPFDFQAEYGLSNQLAEAISDHYPVEVTLR--------------------- | 282 |
| | | +VVP S+ FDFQ Y LS + A +SDH+PVE L+ | |
| Sbjct | 249 | VNSVVPRSSGVFDFQKAYDLSEEEALDVSDHFPVEFKLQSSPAFTNNRKSVSLKKRKKGN | 308 |
| Query | 283 | KISSTMVGSGCKPCICTVPEVSSVFIFPPKPKDVLYITLEPKVTCVVVDISKDDPEVQES | 342 |
| | | + SSTMVGSGCKPCICTVPEVSSVEIFPPKPKDVLYITLEPKVTCVVVDISKDDPEVQES | |
| Sbjct | 309 | RSSSTMVGSGCKPCICTVPEVSSVEIFPPKPKDVLYITLEPKVTCVVVDISKDDPEVQES | 368 |
| Query | 343 | WFVDDVEVHTAQTQPREEQFNSTFRSVSELPIMHQDWINGKEFKCRVNSAAFPAPIEKTI | 402 |
| | | WFVDDVEVHTAQTQPREEQFNSTERSVSELPIMHQDWINGKEFKCRVNSAAFPAPIEKTI | |
| Sbjct | 369 | WFVDDVEVHTAQTQPREEQFNSTERSVSELPIMHQDWINGKEFKQRVNSAAFPAPIEKTI | 428 |
| Query | 403 | SKTKGRPKAPQVYTIPPPKEQMAKDKVSLTCMITDFFPEDITVEWQWNGQPAENYKNTQP | 462 |
| | | SKTKGRPKAPQVYTIPPPKEQMAKDKVSLTCMITDFFPEDITVEWQWNGQPAENYKNTQP | |
| Sbjct | 429 | SKTKGRPKAPQVYTIPPPKEQMAKDKVSLTCMITDFFPEDITVEWQWNGQPAENYKNTQP | 488 |
| Query | 463 | IMDTDGSYFVYSKINVQKSNWEAGNTFTCSVLHEGLANHHTEKSLSHSPGK* | 514 |
| | | IMDTDGSYFVYSKINVQKSNWEAGNTFTCSVLHEGLANARTEKSLSHSPGK* | |
| Sbjct | 489 | IMDTDGSYFVYSKINVQKSNWEAGNTFTCSVLHEGLANHHTEKSLSHSPGK* | 540 |

As used herein, the term “Fc” refers to a human IgG (immunoglobulin) Fc domain. Subtypes of IgG such as IgG1, IgG2, IgG3, and IgG4 are contemplated for usage as Fc domains.

As used herein, the “Fc region” is the portion of an IgG molecule that correlates to a crystallizable fragment obtained by papain digestion of an IgG molecule. The Fc region comprises the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc fragment contains the entire second constant domain CH2 (residues 231-340 of human IgG1, according to the Kabat numbering system) and the third constant domain CH3 (residues 341-447). The term “IgG hinge-Fc region” or “hinge-Fc fragment” refers to a region of an IgG molecule consisting of the Fc region (residues 231-447) and a hinge region (residues 216-230) extending from the N-terminus of the Fc region. The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

As used herein, the term “Fc receptors” refer to proteins found on the surface of certain cells (including, among others, B lymphocytes, follicular dendritic cells, natural killer cells, macrophages, neutrophils, eosinophils, basophils, human platelets, and mast cells) that contribute to the protective functions of the immune system. Fc receptors bind to antibodies that are attached to infected cells or invading pathogens. Immunoglobulin Fc receptors (FcRs) are expressed on all hematopoietic cells and play crucial roles in antibody-mediated immune responses. Binding of immune complexes to FcR activates effector cells, leading to phagocytosis, endocytosis of IgG-opsonized particles, releases of inflammatory mediators, and antibody-dependent cellular cytotoxicity (ADCC). Fc receptors have been described for all classes of immunoglobulins: FcγR and neonatal FcR (FcRn) for IgG, FcεR for IgE, FcαR for IgA, FcδR for IgD and FcμR for IgM. All known Fc receptors structurally belong to the immunoglobulin superfamily, except for FcRn and FcεRII, which are structurally related to class I Major Histocompatibility antigens and C-type lectins, respectively (Fc Receptors, Neil A. Fangera, et al., in Encyclopedia of Immunology (2^{nd} Edition), 1998).

As used herein, the term “FcRn Receptor” refers to the neonatal Fc receptor (FcRn), also known as the Brambell receptor, which is a protein that in humans is encoded by the FCGRT gene. An FcRn specifically binds the Fc domain of an antibody. FcRn extends the half-life of IgG and serum albumin by reducing lysosomal degradation in endothelial cells. IgG, serum albumin, and other serum proteins are continuously internalized through pinocytosis. Generally, serum proteins are transported from the endosomes to the lysosome, where they are degraded. FcRn-mediated transcytosis of IgG across epithelial cells is possible because FcRn binds IgG at acidic pH (<6.5) but not at neutral or higher pH. IgG and serum albumin are bound by FcRn at the slightly acidic pH (<6.5), and recycled to the cell surface where they are released at the neutral pH (>7.0) of blood. In this way IgG and serum albumin avoid lysosomal degradation.

The Fc portion of an IgG molecule is located in the constant region of the heavy chain, notably in the CH2 domain. The Fc region binds to an Fc receptor (FcRn), which is a surface receptor of a B cell and also proteins of the complement system. The binding of the Fc region of an IgG molecule to an FcRn activates the cell bearing the receptor and thus activates the immune system. The Fc residues critical to the mouse Fc-mouse FcRn and human Fc-human FcRn interactions have been identified (Dall'Acqua et al., 2002, J. Immunol. 169(9):5171-80). An FcRn binding domain comprises the CH2 domain (or a FcRn binding portion thereof) of an IgG molecule.

As used herein, the term “fragment,” as applied to a nucleic acid, refers to a subsequence of a larger nucleic acid. A “fragment” of a nucleic acid can be at least about 15, 50-100, 100-500, 500-1000, 1000-1500 nucleotides, 1500-2500, or 2500 nucleotides (and any integer value in between). As used herein, the term “fragment,” as applied to a protein or peptide, refers to a subsequence of a larger protein or peptide, and can be at least about 20, 50, 100, 200, 300 or 400 amino acids in length (and any integer value in between).

The term “functional equivalent” or “functional derivative” denotes, in the context of a functional derivative of an amino acid sequence, a molecule that retains a biological activity (either function or structural) that is substantially similar to that of sequences of DNAse1-Fc and/or DNAse1 E3-Fc constructs shown herein. A functional derivative or equivalent may be a natural derivative or is prepared synthetically. The functionally-equivalent polypeptides of the disclosure can also be polypeptides identified using one or more techniques of structural and or sequence alignment known in art.

Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like. Typically, greater than 30% identity between two polypeptides is considered to be an indication of functional equivalence. Preferably, functionally equivalent polypeptides of the disclosure have a degree of sequence identity with the DNAse1-Fc and/or DNAse1L3-Fc constructs of greater than 80%. More preferred polypeptides have degrees of identity of greater than 85%, 90%, 95%, 98% or 99%, respectively. Method for determining whether a functional equivalent or functional derivative has the same or similar or higher biological activity than the DNAse1-Fc and/or DNAse1L3-Fc construct can be determined by using enzymology assays known in the art.

“Gene transfer” and “gene delivery” refer to methods or systems for reliably inserting a particular nucleic acid sequence into targeted cells.

An “inducible” promoter is a nucleotide sequence that, when operably linked with a polynucleotide that encodes or specifies a gene product, causes the gene product to be produced in a cell substantially only when an inducer that corresponds to the promoter is present in the cell.

As used herein, the term “in vivo half-life” for a protein and/or polypeptide contemplated within the disclosure (such as, for example, a DNAse1 and/or DNAse1L3 construct containing FcRn binding sites) refers to the time required for half the quantity administered in the animal to be cleared from the circulation and/or other tissues in the animal. When a clearance curve of a fusion protein is constructed as a function of time, the curve is usually biphasic with a rapid α-phase (which represents an equilibration of the administered molecules between the intra- and extra-vascular space and which is, in part, determined by the size of molecules), and a longer β-phase (which represents the catabolism of the molecules in the intravascular space). In certain embodiments, the term “in vivo half-life” in practice corresponds to the half-life of the molecules in the β-phase.

“Instructional material,” as that term is used herein, includes a publication, a recording, a diagram, or any other medium of expression that can be used to communicate the usefulness of the nucleic acid, peptide, and/or compound of the disclosure in the kit for identifying or alleviating or treating the various diseases or disorders recited herein.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences that are normally adjacent to the fragment, i.e., the sequences adjacent to the fragment in a genome in which it naturally occurs. The term also applies to nucleic acids that have been substantially purified from other components which naturally accompany the nucleic acid, i.e., RNA or DNA or proteins, which naturally accompany it in the cell. The term therefore includes, for example, a recombinant DNA that is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA that is part of a hybrid gene encoding additional polypeptide sequence.

An “oligonucleotide” or “polynucleotide” is a nucleic acid ranging from at least 2, in certain embodiments at least 8, 15 or 25 nucleotides in length, but may be up to 50, 100, 1000, or 5000 nucleotides long or a compound that specifically hybridizes to a polynucleotide.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the term “patient,” “individual” or “subject” refers to a human.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any one of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, the term “polypeptide” refers to a polymer composed of amino acid residues, related naturally occurring structural variants, and synthetic non-naturally occurring analogues thereof linked via peptide bonds.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements that are required for expression of the gene product. The promoter/regulatory sequence may for example be one that expresses the gene product in a tissue specific manner.

The term “recombinant polypeptide” as used herein is defined as a polypeptide produced by using recombinant DNA methods.

The term “recombinant DNA” as used herein is defined as DNA produced by joining pieces of DNA from different sources.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

As used herein, the term “signal peptide” refers to a sequence of amino acid residues (ranging in length from, for example, 10-30 residues) bound at the amino terminus of a nascent protein of interest during protein translation. The signal peptide is recognized by the signal recognition particle (SRP) and cleaved by the signal peptidase following transport at the endoplasmic reticulum. (Lodish, et al., 2000, Molecular Cell Biology, 4^{th} edition).

As used herein, “substantially purified” refers to being essentially free of other components. For example, a substantially purified polypeptide is a polypeptide that has been separated from other components with which it is normally associated in its naturally occurring state. Non-limiting embodiments include 95% purity, 99% purity, 99.5% purity, 99.9% purity and 100% purity.

A “tissue-specific” promoter is a nucleotide sequence that, when operably linked with a polynucleotide encodes or specified by a gene, causes the gene product to be produced in a cell substantially only if the cell is a cell of the tissue type corresponding to the promoter.

The phrase “under transcriptional control” or “operatively linked” as used herein means that the promoter is in the correct location and orientation in relation to a polynucleotide to control the initiation of transcription by RNA polymerase and expression of the polynucleotide.

The term “transfected” or “transformed” or “transduced” as used herein refers to a process by which exogenous nucleic acid is transferred or introduced into the host cell. A “transfected” or “transformed” or “transduced” cell has been transfected, transformed or transduced with exogenous nucleic acid. The cell includes the primary subject cell and its progeny.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder, or a symptom of a disease or disorder, with the purpose to cure, heal, alleviate, relieve, alter, remedy, ameliorate, improve or affect the disease or disorder, or the symptoms of the disease or disorder. Such treatments may be specifically tailored or modified, based on knowledge obtained from the field of pharmacogenomics.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide may differ in amino acid sequence by one or more substitutions, additions, or deletions in any combination. A variant of a nucleic acid or peptide may be a naturally occurring such as an allelic variant, or may be a variant that is not known to occur naturally. Non-naturally occurring variants of nucleic acids and peptides may be made by mutagenesis techniques or by direct synthesis.

A “vector” is a composition of matter that comprises an isolated nucleic acid and that may be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “virus” is defined as a particle consisting of nucleic acid (RNA or DNA) enclosed in a protein coat, with or without an outer lipid envelope, which is capable of transfecting the cell with its nucleic acid.

As used herein, the term “wild-type” refers to a gene or gene product isolated from a naturally occurring source. A wild-type gene is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. Naturally occurring mutants can be isolated; these are identified by the fact that they have altered characteristics (including altered nucleic acid sequences) when compared to the wild-type gene or gene product.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Constructs and Polypeptides

In one aspect, the disclosure provides a DNAse1-Fc and/or DNAse1L3-Fc construct. The disclosure contemplates that the constructs contemplated herein can have one or more of the mutations described herein.

Further, the disclosure provides homodimeric constructs comprising two independently selected DNAse1 constructs of the disclosure. Further, the disclosure provides homodimeric constructs comprising two independently selected DNAse1L3 constructs of the disclosure. Further, the disclosure provides heterodimeric constructs comprising a DNAse1 construct of the disclosure and a DNAse1L3 construct of the disclosure.

The disclosure provides the constructs described herein, as well as any glycosylation variants (alternative glycoforms), as well as constructs that have been modified through site-directed mutagenesis or any sort of protein chemistry manipulation so as to have improved solubility and/or enzymatic activity and/or in vivo half-life.

In certain embodiments, the construct comprises the amino acid sequence:

DNAse1-X1-LINKER-Fc-X2 (I)

wherein:
DNAse1 is a human DNAse1 polypeptide as described elsewhere herein;
X1 is a covalent bond, or X1 is the peptide of amino acid sequence RAFTNNRKSVSLKKRKKGNRS (SEQ ID NO:3) or a fragment thereof;
LINKER is a chemical bond or a polypeptide comprising 1-100 amino acids;
X2 is null, or X2 is the peptide of amino acid sequence RAFTNNRKSVSLKKRKKGNRS (SEQ ID NO:3) or a fragment thereof;
Fc is the Fc domain of human IgG1 as described elsewhere herein.

In certain embodiments, (I) describes the construct from left to right as from its N-terminus to its C-terminus. In that case, the N-terminus of the Fc is linked to the C-terminus of the DNAse1. In certain embodiments, (I) describes the construct from left to right as from its C-terminus to its N-terminus. In that case, the C-terminus of the Fc is linked to the N-terminus of the DNAse1

In certain embodiments, the polypeptide comprises the amino acid sequence:

DNAse1L3-X1-LINKER-Fc-X2 (II)

wherein:
DNAse1L3 is a human polypeptide DNAse1L3 as described elsewhere herein;
X1 is a covalent bond, or X1 is the peptide of amino acid sequence RAFTNNRKSVSLKKRKKGNRS (SEQ ID NO:3) or a fragment thereof;
LINKER is a covalent bond or a polypeptide comprising 1-100 amino acids;
X2 is null, or X2 is the peptide of amino acid sequence RAFTNNRKSVSLKKRKKGNRS (SEQ ID NO:3) or a fragment thereof;
Fc is the Fc domain of human IgG1 as described elsewhere herein.

In certain embodiments, (II) describes the construct from left to right as from its N-terminus to its C-terminus. In that case, the N-terminus of the Fc is linked to the C-terminus of the DNAse1 L3. In certain embodiments, (II) describes the construct from left to right as from its C-terminus to its N-terminus. In that case, the C-terminus of the Fc is linked to the N-terminus of the DNAse1L3.

### Fc:

In certain embodiments, the Fc domain of human IgG1 has the following sequence:

hIgG Fe domain, Fc (human)
SEQ ID NO: 4
DKTHTCPPCPAPELLGGPSVFLFPPKPKDTIMISRTPEVTCVVVDVSHED
PEVKENWYVDGVEVHNAKTKPREEQYNSTYRVVSVLTVLHQDWINGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGK

In certain embodiments, the Fc domain of mouse IgG1 has the following sequence:

hIgG Fc domain, Fc (mouse)
SEQ ID NO: 31
GCKPCICTVPEVSSVFIFPPKPKDVLYITLEPKVTCVVVDISKDDPEVQF
SWFVDDVEVHTAQTQPREEQFNSTFRSVSELPIMHQDWINGKEFKCRVNS
AAFPAPIEKTISKTKGRPKAPQVYTIPPPKEQMAKDKVSLTCMITDFFPE
DITVEWQWNGQPAENYKNTQPIMDTDGSYFVYSKLNVQKSNWEAGNTFTC
SVLHEGLHNHHTEKSLSHSPGK

In certain embodiments, Cys6 (C6) with respect to SEQ ID NO:4 is mutated to another amino acid, such as but not limited to G or S. In certain embodiments, Cys9 (C9) with respect to SEQ ID NO:4 is mutated to another amino acid, such as but not limited to Gly or Ser. In a non-limiting embodiment, any one of such mutations in the C6/C9 residues responsible for the interchain disulfide bond in the heavy chain of the Fc domain converts a dimeric enzyme fusion to a monomeric fusion, thus allowing for greater accessibility to chromatin and microparticle DNA.

In certain embodiments, the hIgG Fc domain has at least one of the following mutations with respect to SEQ ID NO:4: M32Y, S34T, and T36E. In a non-limiting embodiment, any such mutations enhances endosomal recycling of the corresponding construct. In certain embodiments, the hIgG Fc domain has the following mutations with respect to SEQ ID NO:4: M32Y, S34T, and T36E.

A non-limiting list of contemplated mutations in the Fc domain of the constructs of the disclosure include C6S, C9S, M32Y, S34T, and/or T36E with respect to SEQ ID NO:4. In certain embodiments, the Fc domain of the construct comprise the C6S mutation with respect to SEQ ID N0:4. In certain embodiments, the Fc domain of the construct comprise the C9S mutation with respect to SEQ ID NO:4. In certain embodiments, the Fc domain of the construct comprise the M32Y mutation with respect to SEQ ID NO:4. In certain embodiments, the Fc domain of the construct comprise the S34T mutation with respect to SEQ ID NO:4. In certain embodiments, the Fc domain of the construct comprise the T36E mutation with respect to SEQ ID NO:4.

### Linker:

In certain embodiments, the LINKER is a chemical bond or absent. In certain embodiments, the LINKER is a polypeptide comprising 1-100, 1-90, 1-80, 1-70, 1-60, 1-50, 1-40, 1-30, 1-20, 1-10, and/or 1-5 amino acids. In certain embodiments, the LINKER comprises Gly and/or Ser amino acids.

In certain embodiments, the LINKER comprises GS. In certain embodiments, the LINKER comprises GSC. In certain embodiments, the LINKER comprises GGGGSGGGGS (SEQ ID NO:5). In certain embodiments, the LINKER comprises SSTMVRS (SEQ ID NO:40). In certain embodiments, the LINKER comprises SSTMVGS (SEQ ID NO:41).

In certain embodiments, the LINKER comprises ELKTPLGDTTHTXPRZPAPELLGGP (SEQ ID NO:6), wherein each occurrence of X is C, G, or S, and wherein each occurrence of Z is C, G, or S. In certain non-limiting embodiments, at least one of X and Z is not C and formation of a disulfide bridge is prevented. In certain embodiments, SEQ ID NO:6 corresponds to the hinge region of Human IgG1.

### X1 and X2:

In certain embodiments, X1 is a covalent bond. In certain embodiments, X1 is the peptide of amino acid sequence RAFTNNRKSVSLKKRKKGNRS (SEQ ID NO:3) or a fragment thereof.

In certain embodiments, X2 is a covalent bond. In certain embodiments, X2 is the peptide of amino acid sequence RAFTNNRKSVSLKKRKKGNRS (SEQ ID NO:3) or a fragment thereof.

### DNAse1:

An illustrative construct of the disclosure comprises the amino acid sequence of SEQ ID NO:7, wherein the bold sequence corresponds to the DNAse1 polypeptide, wherein the underlined sequence corresponds to the Fc, and wherein the italics sequence corresponds to the LINKER.

SEQ ID NO: 7
MRGMKLLGALLALAALLQGAVSLKIAAFNIQTFGETKMSNATLVSYIVQI
LSRYDIALVQEVRDSHLTAVGKLLDNLNQDAPDTYHYVVSEPLGRNSYKE
RYLFVYRPDQVSAVDSYYYDDGCEPCGNDTFNREPAIVRFFSRFTEVREF
AIVPLHAAPGDAVAEIDALYDVYLDVQEKWGLEDVMLMGDENAGCSYVRP
SQWSSIRLWTSPTFQWLIPDSADTTATPTHCAYDRIVVAGMLLRGAVVPD
SALPENFQAAYGLSDQLAQAISDHYPVEVMLKGSDKTHTCPPCPAPELLG
GPSVFLFPPKPKDTLMISRTPEVTCVVVDVSHEDPEVKENWYVDGVEVHN
AKTKPREEQYNSTYRVVSVLTVLHQDWINGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPGK

In certain embodiments, the construct has one or more of the following mutations in Fc: C290S, C293S, M316Y, S318T, and/or T320E with respect to SEQ ID NO:7.

An illustrative construct of the disclosure comprises the amino acid sequence of SEQ ID NO:8, wherein the bold sequence corresponds to the DNAse1 polypeptide, wherein the underlined sequence corresponds to the Fc, and wherein the italics sequence corresponds to the LINKER.

SEQ ID NO: 8
MRGMKLLGALLALAALLQGAVSLKIAAFNIQTFGETKMSNATLVSYIVQI
LSRYDIALVQEVRDSHLTAVGKLLDNLNQDAPDTYHYVVSEPLGRNSYKE
RYLFVYRPDQVSAVDSYYYDDGCEPCGNDTFNREPAIVRFFSRFTEVREF
AIVPLHAAPGDAVAEIDALYDVYLDVQEKWGLEDVMLMGDENAGCSYVRP
SQWSSIRLWTSPTFOWLIPDSADTTATPTHCAYDRIVVAGMLLRGAVVPD
SALPENFQAAYGLSDQLAQAISDHYPVEVMLKGSDKTHISPPSPAPELLG
GPSVFLFPPKPKDTLYITREPEVTCVVVDVSHEDPEVKENWYVDGVEVHN
AKTKPREEQYNSTYRVVSVLTVLHQDWLNGKEYKCKVSNKALPAPIEKTI
SKAKGQPREPQVYTIPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPGK

In certain embodiments, the construct lacks at least a portion of the signal peptide of DNAse1 corresponding to residues 1-22 of SEQ ID NO:1. In certain embodiments, the construct lacks the signal peptide of DNAse1 corresponding to residues 1-22 of SEQ ID NO:1.

A non-limiting list of contemplated mutations in the DNAse1 domain of the constructs of the disclosure with respect to SEQ ID NO:1 include but are not limited to Q31R, E35R, Y46H, Y46S, V88N, N96K, D109N, V111T, A136F, R148S, E149N, M186I, L208P, D220N, D250N, A252T, G262N, D265N, and L267T.

In certain embodiments, the DNAse1 domain of the construct comprises the mutation Q31R with respect to SEQ ID NO:1. In certain embodiments, the DNAse1 domain of the construct comprises the mutation E35R with respect to SEQ ID NO:1. In certain embodiments, the DNAse1 domain of the construct comprises the mutation Y46H with respect to SEQ ID NO:1. In certain embodiments, the DNAse1 domain of the construct comprises the mutation Y46S with respect to SEQ ID NO:1. In certain embodiments, the DNAse1 domain of the construct comprises the mutation V88N with respect to SEQ ID NO:1. In certain embodiments, the DNAse1 domain of the construct comprises the mutation N96K with respect to SEQ ID NO:1. In certain embodiments, the DNAse1 domain of the construct comprises the mutation D109N with respect to SEQ ID NO:1. In certain embodiments, the DNAse1 domain of the construct comprises the mutation V111T with respect to SEQ ID NO:A. In certain embodiments, the DNAse1 domain of the construct comprises the mutation A136F with respect to SEQ ID NO:1. In certain embodiments, the DNAse1 domain of the construct comprises the mutation R148S with respect to SEQ ID NO:1. In certain embodiments, the DNAse1 domain of the construct comprises the mutation E149N with respect to SEQ ID NO:1. In certain embodiments, the DNAse1 domain of the construct comprises the mutation M186I with respect to SEQ ID NO:1. In certain embodiments, the DNAse1 domain of the construct comprises the mutation L208P with respect to SEQ ID NO:1. In certain embodiments, the DNAse1 domain of the construct comprises the mutation D220N with respect to SEQ ID NO:1. In certain embodiments, the DNAse1 domain of the construct comprises the mutation D250N with respect to SEQ ID NO:1. In certain embodiments, the DNAse1 domain of the construct comprises the mutation A252T with respect to SEQ ID NO:1. In certain embodiments, the DNAse1 domain of the construct comprises the mutation G262N with respect to SEQ ID NO:1. In certain embodiments, the DNAse1 domain of the construct comprises the mutation D265N with respect to SEQ ID NO:1. In certain embodiments, the DNAse1 domain of the construct comprises the mutation L267T with respect to SEQ ID NO:1.

In certain non-limiting embodiments, the mutation A136F with respect to SEQ ID NO:1 decreases actin binding of the construct.

In certain non-limiting embodiments, the mutation(s) E35R, Y46H, Y46S, R148S, E149N, M186I, L208P, and/or D220N increase the enzymatic activity of the construct.

In certain non-limiting embodiments, the mutation(s) V88N, D109N, V111T, G262N, D265N, and/or L267T modify the overall glycosylation status of the construct.

Non-limiting examples of constructs of the disclosure comprise the following amino acid sequences, wherein the bold sequence corresponds to the DNAse1 polypeptide, wherein the underlined sequence corresponds to the Fc, wherein the italics sequence corresponds to the LINKER, and wherein the italics/underlined sequence corresponds to X1/X2. Certain mutations are shown as doubly underlined.

SEQ ID NO: 9
MRGMKLLGALLALAALLQGAVSLKIAAFNIQTFGRTKMSNATLVSYIVQILSRYDIALVQEVRD
SHLTAVGKLLDNLNQDAPDTYHYNVSEPLGRNSYKERYLFVYRPNQTSAVDSYYYDDGCEPCGN
DTFNREPFIVRFFSRFTEVREFAIVPLHAAPGDAVAEIDALYDVYLDVQEKWGLEDVMLMGDEN
AGCSYVRPSQWSSIRLWTSPTFQWLIPDSADTTATPTHCAYDRIVVAGMLLRGAVVPDSALPEN
FQAAYNLSNQTAQAISDHYPVEVMLKGSDKTHTSPPSPAPELLGGPSVFLFPPKPKDTLYITRE
PEVTCVVVDVSHEDPEVKENWYVDGVEVHNAKTKPREEQYNSTYRVVSVLTVLHQDWLNGKEYK
CKVSNKALPAPIEKTISKAKGOPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKSLSLSPGK
SEQ ID NO: 10
MRGMKLLGALLALAALLQGAVSLKIAAFNIQTFGETKMSNATLVSYIVQILSRYDIALVQEVRD
SHLTAVGKLLDNLNODAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDEN
AGCSYVRPSQWSSIRLWTSPTFQWLIPDSADTTATPTHCAYDRIVVAGMLLRGAVVPDSALPEN
FQAAYGLSDQLAQAISDHYPVEVMLKRAFTNNRKSVSLKKRKKGNRSGSDKTHISPPSPAPELL
GGPSVFLFPPKPKDTLYITREPEVTCVVVDVSHEDPEVKENWYVDGVEVHNAKTKPREEQYNST
YRVVSVLTVLHQDWLNGKEYKCKVSNKALPAPIEKTISKAKGQPREPQVYTLPPSREEMTKNQV
SLICLVKGFYPSDIAVEWESNGQPENNYKTTPPVLDSDGSFFLYSKLTVDKSRWQQGNVFSCSV
MHEALHNHYTQKSLSLSPGKRAFTNNRKSVSLKKRKKGNRS
SEQ ID NO: 11
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDEN
AGCSYVRPSQWSSIRLWTSPTFQWLIPDSADTTATPTHCAYDRIVVAGMLLRGAVVPDSALPEN
FQAAYGLSDQLAQAISDHYPVEVMLKGSDKTHTSPPSPAPELLGGPSVFLFPPKPKDTLYITRE
PEVTCVVVDVSHEDPEVKENWYVDGVEVHNAKTKPREEQYNSTYRVVSVLIVLHQDWLNGKEYK
CKVSNKALPAPIEKTISKAKGQPREPQVYTIPPSREEMTKNQVSLTCLVKGFYPSDIAVEWESN
GQPENNYKTTPPVLDSDGSFFLYSKLTVDKSRWQQGNVFSCSVMHEALHNHYTQKSLSLSPGKR
AFTNNRKSVSLKKRKKGNRS
SEQ ID NO: 12
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDEN
AGCSYVRPSQWSSIRLWTSPTFQWLIPDSADTTATPTHCAYDRIVVAGMLLRGAVVPDSALPEN
FQAAYGLSDQLAQAISDHYPVEVMLKRAFTNNRKSVSLKKRKKGNRSGSDKTHTSPPSPAPELL
GGPSVFLFPPKPKDTLYITREPEVTCVVVDVSHEDPEVKENWYVDGVEVHNAKIKPREEQYNST
YRVVSVLIVLHQDWINGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNVESCSV
MHEALHNHYTQKSLSLSPGK
SEQ ID NO: 13
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDEN
AGCSYVRPSQWSSIRLWTSPTFQWLIPDSADTTATPTHCAYDRIVVAGMLLRGAVVPDSALPFN
FQAAYGLSDQLAQAISDHYPVEVMLKGGGGSGGGGSDKTHTSPPSPAPELLGGPSVFLFPPKPK
DTLYITREPEVTCVVVDVSHEDPEVKENWYVDGVEVHNAKIKPREEQYNSTYRVVSVLTVLHQD
WLNGKEYKCKVSNKALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSVMHEALHNHYTQKS
LSLSPGK
SEQ ID NO: 14
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVPDSALPFN
FQAAYGLSDQLAQAISDHYPVEVMLKELKTPLGDTTHTXPRZPAPELLGGPDKTHTSPPSPAPE
LLGGPSVELFPPKPKDTLYITREPEVTCVVVDVSHEDPEVKENWYVDGVEVHNAKTKPREEQYN
STYRVVSVLIVLHQDWINGKEYKCKVSNKALPAPIEKTISKAKGQPREPQVYTLPPSREEMTKN
QVSLTCLVKGFYPSDIAVEWESNGQPENNYKITPPVLDSDGSFFLYSKLTVDKSRWQQGNVFSC
SVMHEALHNHYTQKSLSLSPGK

wherein X and Z are independently Cys, Gly, or Ser.

In certain non-limiting embodiments, wherein at least one of X and Z is not Cys (C) formation of disulfide bridge is prevented.

SEQ ID NO: 15
MRGMKLLGALLALAALLQGAVSLKIAAFNI [symbol] TFGETKMSNATLVSYIVQI
LSRYDIALVQEVRDSHLTAVGKLLDNLNQDAPDTYHYVVSEPLGR [symbol] SYKE
RYLFVYRPDQVSAVDSYYYDDGCEPCGNDTFNREP [symbol] IVRFFSRFTEVREF
AIVPLHAAPGDAVAEIDALYDVYLDVQEKWGLEDVMLMGDFNAGCSYVRP
SQWSSIRLWTSPTFQWLIPDSADTTATPTHCAYDRIVVAGMLLQGAVVPD
SALPENFQAAYGLSDQLAQAISDHYPVEVMLKGSCDKTHTCPPCPAPELL
GGPSVFLFPPKPKDTLYITREPEVTCVVVDVSHEDPEVKENWYVDGVEVH
NAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNH
YTQKSLSLSPGK
SEQ ID NO: 16
MRGMKLLGALLALAALLQGAVSLKIAAFNI [symbol] TFGETKMSNATLVSYIVQI
LSRYDIALVQEVRDSHLTAVGKLLDNLNQDAPDTYHYVVSEPLGR [symbol] SYKE
RYLFVYRPDQVSAVDSYYYDDGCEPCGNDTFNREP [symbol] IVRFFSRFTEVREF
AIVPLHAAPGDAVAEIDALYDVYLDVQEKWGLEDVMLMGDENAGCSYVRP
SQWSSIRLWTSPTFQWLIPDSADTTATPTHCAYDRIVVAGMLLQGAVVPD
SALPENFQAAYGLS [symbol] Q [symbol] AQAISDHYPVEVMLKGSCDKTHTCPPCPAPELL
GGPSVFLFPPKPKDTLYITREPEVTCVVVDVSHEDPEVKENWYVDGVEVH
NAKTKPREEQYNSTYRVVSVLIVLHQDWINGKEYKCKVSNKALPAPIEKT
ISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNH
YTQKSLSLSPGK
SEQ ID NO: 17
MRGMKLLGALLALAALLQGAVSLKIAAFNI [symbol] TFGETKMSNATLVSYIVQI
LSRYDIALVQEVRDSHLTAVGKLLDNLNQDAPDTYHYVVSEPLGR [symbol] SYKE
RYLFVYRPDQVSAVDSYYYDDGCEPCGNDTFNREP [symbol] IVRFFSRFTEVREF
AIVPLHAAPGDAVAEIDALYDVYLDVQEKWGLEDVMLMGDENAGCSYVRP
SQWSSIRLWTSPTFQWLIPDSADTTATPTHCAYDRIVVAGMLLQGAVVP [symbol]
S [symbol] LPFNFQAAYGLSDQLAQAISDHYPVEVMLKGSCDKTHTCPPCPAPELL
GGPSVFLFPPKPKDTLYITREPEVTCVVVDVSHEDPEVKENWYVDGVEVH
NAKTKPREEQYNSTYRVVSVLTVLHQDWINGKEYKCKVSNKALPAPIEKT
ISKAKGQPREPQVYTLPPSREEMTKNQVSLTCLVKGFYPSDIAVEWESNG
QPENNYKTIPPVLDSDGSFFLYSKLTVDKSRWQQGNVFSCSVMHEALHNH
YTQKSLSLSPGK

### DNAse1L3:

An illustrative construct of the disclosure comprises the amino acid sequence of SEQ ID NO:18, wherein the bold sequence corresponds to the DNAse1L3 polypeptide, wherein the underlined sequence corresponds to the Fc, and wherein the italics sequence corresponds to the LINKER.

SEQ ID NO: 18
MSRELAPLLLLLLSIHSALAMRICSFNVRSFGESKQEDKNAMDVIVKVIK
RCDIILVMEIKDSNNRICPILMEKLNRNSRRGITYNYVISSRLGRKTYKE
QYAFLYKEKLVSVKRSYHYHDYQDGDADVFSREPFVVWFQSPHTAVKDFV
IIPLHTTPETSVKEIDELVEVYTDVKHRWKAENFIFMGDFNAGCSYVPKK
AWKNIRLRTDPRFVWLIGDQEDTTVKKSTNCAYDRIVLRGQEIVSSVVPK
SNSVFDFQKAYKLTEEEALDVSDHFPVEFKLQSSRAFTNSKKSVTLRKKT
KSKRSGSDKTHTCPPCPAPELLGGPSVFLFPPKPKDTLMISRTPEVTCVV
VDVSHEDPEVKENWYVDGVEVHNAKTKPREEQYNSTYRVVSVLTVLHQDW
INGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVD
KSRWQQGNVESCSVMHEALHNHYTQKSLSLSPGK

In certain embodiments, the construct has one or more of the following mutations in Fc: C313S, C316S, M339Y, S341T, and/or T342E with respect to SEQ ID NO:18.

An illustrative construct of the disclosure comprises the amino acid sequence of SEQ ID NO:19, wherein the bold sequence corresponds to the DNAse1L3 polypeptide, wherein the underlined sequence corresponds to the Fc, and wherein the italics sequence corresponds to the LINKER.

SEQ ID NO: 19
MSRELAPLLLLLLSIHSALAMRICSFNVRSFGESKQEDKNAMDVIVKVIK
RCDIILVMEIKDSNNRICPILMEKLNRNSRRGITYNYVISSRLGRKTYKE
QYAFLYKEKLVSVKRSYHYHDYQDGDADVFSREPFVVWFQSPHTAVKDFV
IIPLHTTPETSVKEIDELVEVYTDVKHRWKAENFIFMGDFNAGCSYVPKK
AWKNIRLRTDPRFVWLIGDQEDTTVKKSINCAYDRIVLRGQEIVSSVVPK
SNSVFDFQKAYKLTEEEALDVSDHFPVEFKLQSSRAFTNSKKSVTLRKKT
KSKRSGSDKTHTSPPSPAPELLGGPSVFLFPPKPKDTLYITREPEVTCVV
VDVSHEDPEVKENWYVDGVEVHNAKTKPREEQYNSTYRVVSVLTVLHQDW
INGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVD
KSRWQQGNVFSCSVMHEALHNHYTQKSLSLSPGK

In certain embodiments, the construct lacks at least a portion of the signal peptide of DNAse1L3 corresponding to residues 1-20 of SEQ ID NO:2. In certain embodiments, the construct lacks the signal peptide of DNAse1L3 corresponding to residues 1-20 of SEQ ID NO:2.

In certain embodiments, the construct lacks at least a portion of the nuclear localization sequence (NLS) of the DNAse1L3 polypeptide. In certain embodiments, the construct lacks residues 291-305 of SEQ ID NO:2. In certain embodiments, the construct lacks residues 292-304 of SEQ ID NO:2. In certain embodiments, the construct lacks residues 296-304 of SEQ ID NO:2. In certain embodiments, the construct lacks residues A-B of SEQ ID NO:2, wherein A ranges from 291 to 296 and B ranges from 304 to 305.

A non-limiting list of contemplated mutations in the Fc domain of the constructs of the disclosure, with respect to SEQ ID NO:18, include C313S, C316S, M339Y, S341T, and/or T342E.

A non-limiting list of contemplated mutations in the DNAse1L3 domain of the constructs of the disclosure, with respect to SEQ ID NO:2, include E33R, M42T, V44H, V88T, N96K, A127N, V129T, K147S, D148N, L207P, D219N, and/or V254T.

In certain embodiments, the DNAse1L3 domain of the construct comprises the mutation E33R with respect to SEQ ID NO:2. In certain embodiments, the DNAse1L3 domain of the construct comprises the mutation M42T with respect to SEQ ID NO:2. In certain embodiments, the DNAse1L3 domain of the construct comprises the mutation V44H with respect to SEQ ID N0:2. In certain embodiments, the DNAse1L3 domain of the construct comprises the mutation V88T with respect to SEQ ID NO:2. In certain embodiments, the DNAse1L3 domain of the construct comprises the mutation N96K with respect to SEQ ID NO:2. In certain embodiments, the DNAse1L3 domain of the construct comprises the mutation A127N with respect to SEQ ID NO:2. In certain embodiments, the DNAse1L3 domain of the construct comprises the mutation V129T with respect to SEQ ID NO:2. In certain embodiments, the DNAse1L3 domain of the construct comprises the mutation K147S with respect to SEQ ID NO:2. In certain embodiments, the DNAse1L3 domain of the construct comprises the mutation D148N with respect to SEQ ID N0:2. In certain embodiments, the DNAse1L3 domain of the construct comprises the mutation L207P with respect to SEQ ID NO:2. In certain embodiments, the DNAse1L3 domain of the construct comprises the mutation D219N with respect to SEQ ID NO:2. In certain embodiments, the DNAse1L3 domain of the construct comprises the mutation V254T with respect to SEQ ID NO:2.

In certain non-limiting embodiments, the mutation A136F with respect to SEQ ID NO:1 decreases actin binding of the construct.

In certain non-limiting embodiments, the mutation(s) E33R, V44H, N96K, K147S, D148N, L207P, and/or D219N with respect to SEQ ID NO:1 increase(s) the enzymatic activity of the construct.

In certain non-limiting embodiments, the mutation V254T modifies the overall glycosylation status of the construct.

Non-limiting examples of constructs of the disclosure comprise the following amino acid sequences, wherein the bold sequence corresponds to the DNAse1L3 polypeptide, wherein the underlined sequence corresponds to the Fc, wherein the italics sequence corresponds to the LINKER, and wherein the italics/underlined sequence corresponds to X1/X2. Certain mutations are shown as doubly underlined.

SEQ ID NO: 20
MSRELAPLLLLLLSIHSALAMRICSFNVRSFGESKQEDKNAMDVIVKVIK
RCDIILVMEIKDSNNRICPILMEKLNRNSRRGITYNYVISSRLGRKTYKE
QYAFLYKEKLVSVKRSYHYHDYQDGDADVFSREPFVVWFQSPHTAVKDFV
IIPLHTTPETSVKEIDELVEVYTDVKHRWKAENFIFMGDFNAGCSYVPKK
AWKNIRLRTDPRFVWLIGDQEDTTVKKSTNCAYDRIVLRGQEIVSSVVPK
SNSVFDFQKAYKLTEEEALDVSDHFPVEFKLQSSRAFTNSKKSVTLRKKT
KSKRSGSDKTHTSPPSPAPELLGGPSVELFPPKPKDTLYITREPEVTCVV
VDVSHEDPEVKENWYVDGVEVHNAKTKPREEQYNSTYRVVSVLIVLHQDW
INGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVD
KSRWQQGNVFSCSVMHEALHNHYTQKSLSLSPGKRAFTNNRKSVSLKKRK
KGNRS
SEQ ID NO: 21
MSRELAPLLLLLLSIHSALAMRICSFNVRSFGESKQEDKNAMDVIVKVIK
RCDIILVMEIKDSNNRICPILMEKLNRNSRRGITYNYVISSRLGRKTYKE
QYAFLYKEKLVSVKRSYHYHDYQDGDADVFSREPFVVWFQSPHTAVKDFV
IIPLHTTPETSVKEIDELVEVYTDVKHRWKAENFIFMGDFNAGCSYVPKK
AWKNIRLRTDPRFVWLIGDQEDTTVKKSINCAYDRIVLRGQEIVSSVVPK
SNSVFDFQKAYKLTEEEALDVSDHFPVEFKLQSSRAFTNSKKSVTLRKKT
KSKRSRAFTNNRKSVSLKKRKKGNRSGSDKTHTSPPSPAPELLGGPSVFL
FPPKPKDTLYITREPEVTCVVVDVSHEDPEVKENWYVDGVEVHNAKTKPR
EEQYNSTYRVVSVLTVLHQDWLNGKEYKCKVSNKALPAPIEKTISKAKGQ
PREPQVYTIPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGKRAFTNNRKSVSLKKRKKGNRS
SEQ ID NO: 22
MSRELAPLLLLLLSIHSALAMRICSFNVRSFGESKQEDKNAMDVIVKVIK
RCDIILVMEIKDSNNRICPILMEKLNRNSRRGITYNYVISSRLGRKTYKE
QYAFLYKEKLVSVKRSYHYHDYQDGDADVFSREPFVVWFQSPHTAVKDFV
IIPLHTTPETSVKEIDELVEVYTDVKHRWKAENFIFMGDFNAGCSYVPKK
AWKNIRLRTDPRFVWLIGDQEDTTVKKSTNCAYDRIVLRGQEIVSSVVPK
SNSVFDFQKAYKLTEEEALDVSDHFPVEFKLQSSRAFTNSKKSVTLRKKT
KSKRSRAFTNNRKSVSLKKRKKGNRSGSDKTHTSPPSPAPELLGGPSVFL
FPPKPKDTLYITREPEVTCVVVDVSHEDPEVKENWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGK
SEQ ID NO: 23
MSRELAPLLLLLLSIHSALAMRICSFNVRSFGESKQEDKNAMDVIVKVIK
RCDIILVMEIKDSNNRICPILMEKLNRNSRRGITYNYVISSRLGRKTYKE
QYAFLYKEKLVSVKRSYHYHDYQDGDADVFSREPFVVWFQSPHTAVKDFV
IIPLHTTPETSVKEIDELVEVYTDVKHRWKAENFIFMGDFNAGCSYVPKK
AWKNIRLRTDPRFVWLIGDQEDTTVKKSTNCAYDRIVLRGQEIVSSVVPK
SNSVFDFQKAYKLTEEEALDVSDHFPVEFKLQSSRAFTNSKKSVTLRKKT
KSKRSGGGGSGGGGSDKTHTSPPSPAPELLGGPSVFLFPPKPKDTLYITR
EPEVTCVVVDVSHEDPEVKENWYVDGVEVHNAKTKPREEQYNSTYRVVSV
LIVLHQDWINGKEYKCKVSNKALPAPIEKTISKAKGQPREPQVYTLPPSR
EEMTKNQVSLTCLVKGFYPSDIAVEWESNGQPENNYKTTPPVLDSDGSFF
LYSKLTVDKSRWQQGNVESCSVMHEALHNHYTQKSLSLSPGK
SEQ ID NO: 24
MSRELAPLLLLLLSIHSALAMRICSFNVRSFGESKQEDKNAMDVIVKVIK
RCDIILVMEIKDSNNRICPILMEKLNRNSRRGITYNYVISSRLGRKTYKE
QYAFLYKEKLVSVKRSYHYHDYQDGDADVFSREPFVVWFQSPHTAVKDFV
IIPLHTTPETSVKEIDELVEVYTDVKHRWKAENFIFMGDFNAGCSYVPKK
AWKNIRLRTDPRFVWLIGDQEDTTVKKSTNCAYDRIVLRGQEIVSSVVPK
SNSVFDFQKAYKLTEEEALDVSDHFPVEFKLQSSRAFTNSKKSVTLRKKT
KSKRSELKTPLGDTTHTXPRZPAPEFLGGPDKTHTXPPZPAPELLGGPSV
FLFPPKPKDTLYITREPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMIKNQVSLTCLVKGFYPSDIAVEWESNGQPENN
YKTTPPVLDSDGSFFLYSKLTVDKSRWQQGNVFSCSVMHEALHNHYTQKS
LSLSPGK*

wherein each occurrence of X and Z is independently Cys, Gly, or Ser.

In certain non-limiting embodiments, wherein at least one of X and Z is not Cys (C) formation of disulfide bridge is prevented.

SEQ ID NO: 25
MSRELAPLLLLLLSIHSALAMRICSFNVRSFGESKQEDKNAMDVIVKVIK
RCDIILVMEIKDSNNRICPILMEKLNRNSRRGITYNYVISSRLGRKTYKE
QYAFLYKEKLVSVKRSYHYHDYQDGDADVFSREPFVVWFQSPHTAVKDFV
IIPLHTTPETSVKEIDELVEVYTDVKHRWKAENFIFMGDFNAGCSYVPKK
AWKNIRLRTDPRFVWLIGDQEDTTVKKSTNCAYDRIVLRGQEIVSSVVPK
SNSVFDFQKAYKLTEEEALDVSDHFPVEFKLQSSRAFTNSGSDKTHTXPP
ZPAPELLGGPSVELFPPKPKDTLYITREPEVTCVVVDVSHEDPEVKENWY
VDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVM
HEALHNHYTQKSLSLSPGK

wherein X and Z are independently C, G, or S.

In certain non-limiting embodiments, wherein at least one of X and Z is not Cys (C) formation of disulfide bridge is prevented.

SEQ ID NO: 26
MSRELAPLLLLLLSIHSALAMRICSFNVRSFG [symbol] SKQEDKNA [symbol] DVIVKVIKRCDIILVMEIKDSN
NRICPILMEKLNRNSRRGITYNYVISSRLGRKTYKEQYAFLYKEKLVSVKRSYHYHDYQDGDAD
VFSREPFVVWFQSPHTAVKDFVIIPLHTTPETSVKEIDELVEVYTDVKHRWKAENFIFMGDFNA
GCSYVPKKAWKNIRLRTDPRFVWLIGDQEDTTVKKSTNCAYDRIVLRGQEIVSSVVPKSNSVFD
FQKAYKLTEEEALDVSDHFPVEFKLQSSRAFTNSKKSVTLRKKTKSKRSGSCDKTHTCPPCPAP
ELLGGPSVFLFPPKPKDILYITREPEVTCVVVDVSHEDPEVKENWYVDGVEVHNAKTKPREEQY
NSTYRVVSVLTVLHQDWLNGKEYKCKVSNKALPAPIEKTISKAKGQPREPQVYTIPPSREEMTK
NQVSLTCLVKGFYPSDIAVEWESNGQPENNYKTTPPVLDSDGSFFLYSKLTVDKSRWQQGNVES
CSVMHEALHNHYTQKSLSLSPGK
SEQ ID NO: 27
MSRELAPLLLLLLSIHSALAMRICSFNVRSFG [symbol] SKQEDKNA [symbol] DVIVKVIKRCDIILVMEIKDSN
NRICPILMEKLNRNSRRGITYNYVISSRLGRKTYKEQYAFLYKEKLVSVKRSYHYHDYQDGD [symbol] D [symbol]
FSREPFVVWFQSPHTAVKDFVIIPLHTTPETSVKEIDELVEVYTDVKHRWKAENFIFMGDFNA
GCSYVPKKAWKNIRLRTDPRFVWLIGDQEDTTVKKSTNCAYDRIVLRGQEIVSSVVPKSNSVFD
FQKAYKLTEEEALDVSDHFPVEFKLQSSRAFTNSKKSVTLRKKTKSKRSGSCDKTHTCPPCPAP
ELLGGPSVFLFPPKPKDTLYITREPEVTCVVVDVSHEDPEVKFNWYVDGVEVHNAKTKPREEQY
NSTYRVVSVLIVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVES
CSVMHEALHNHYTQKSLSLSPGK
SEQ ID NO: 28
MSRELAPLLLLLLSIHSALAMRICSFNVRSFG [symbol] SKQEDKNA [symbol] DVIVKVIKRCDIILVMEIKDSN
NRICPILMEKLNRNSRRGITYNY [symbol] ISSRLGRKTYKEQYAFLYKEKLVSVKRSYHYHDYQDGDAD
VFSREPFVVWFQSPHTAVKDFVIIPLHTTPETSVKEIDELVEVYTDVKHRWKAENFIFMGDFNA
GCSYVPKKAWKNIRLRTDPRFVWLIGDQEDTTVKKSTNCAYDRIVLRGQEIVSSVVPKSNSVFD
FQKAYKLTEEEALDVSDHFPVEFKLQSSRAFTNSKKSVTLRKKTKSKRSGSCDKTHTCPPCPAP
ELLGGPSVFLFPPKPKDTLYITREPEVTCVVVDVSHEDPEVKENWYVDGVEVHNAKTKPREEQY
NSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFS
CSVMHEALHNHYTQKSLSLSPGK
(Sequence 1171 - mouse DNAse1 construct)
SEQ ID NO: 32
MRYTGLMGTLLTLVNLLQLAGTLRIAAFNIRTFGETKMSNATLSVYFVKILSRYDIAVIQEVRD
SHLVAVGKLLDELNRDKPDTYRYVVSEPLGRKSYKEQYLFVYRPDQVSILDSYQYDDGCECGND
TFSREPAIVKFFSPYTEVQEFAIVPLHAAPTEAVSEIDALYDVYLDVWQKWGLEDIMFMGDFNA
GCSYVTSSQWSSIRLRTSPIFQWLIPDSADTTVTSTHCAYDRIVVAGALLQAAVVPNSAVPFDF
QAEYGLSNQLAEAISDHYPVEVTLRKISSTMVRSGCKPCICTVPEVSSVFIFPPKPKDVLYITL
EPKVTCVVVDISKDDPEVQFSWFVDDVEVHTAQTQPREEQENSTERSVSELPIMHQDWLNGKEF
KCRVNSAAFPAPIEKTISKTKGRPKAPQVYTIPPPKEQMAKDKVSLTCMITDFFPEDITVEWQW
NGQPAENYKNTQPIMDTDGSYFVYSKLNVQKSNWEAGNTFTCSVLHEGLHNHHTEKSLSHSPGK
(Sequence 1671 - mouse DNAse1 construct)
SEQ ID NO: 33
MRYTGLMGTLLTLVNLLQLAGTLRIAAFNIRTFGETKMSNATLSVYFVKILSRYDIAVIQEVRD
SHLVAVGKLLDELNRDKPDTYRYVVSEPLGRKSYKEQYLFVYRPDQVSILDSYQYDDGCEPCGN
DTFSREPFIVKFFSPYTEVQEFAIVPLHAAPTEAVSEIDALYDVYLDVWQKWGLEDIMFMGDEN
AGCSYVTSSQWSSIRLRTSPIFQWLIPDSADTTVTSTHCAYDRIVVAGALLQAAVVPNSAVPFD
FQAEYGLSNQLAEAISDHYPVEVTLRKISSTMVGSGCKPCICTVPEVSSVFIFPPKPKDVLYIT
LEPKVTCVVVDISKDDPEVQFSWFVDDVEVHTAQTQPREEQFNSTFRSVSELPIMHQDWLNGKE
FKCRVNSAAFPAPIEKTISKTKGRPKAPQVYTIPPPKEQMAKDKVSLTCMITDFFPEDITVEWQ
WNGQPAENYKNTQPIMDTDGSYFVYSKLNVQKSNWEAGNTFTCSVLHEGLHNHHTEKSLSHSPG
K
(Sequence 1687 - mouse DNAse1 construct)
SEQ ID NO: 34
MRYTGLMGTLLTLVNLLQLAGTLRIAAFNIRTFGRTKMSNATLSVYFVKILSRYDIAVIQEVRD
SHLVAVGKLLDELNRDKPDTYRYNVSEPLGRKSYKEQYLFVYRPDQVSILDSYQYDDGCEPCGN
DTFSREPAIVKFFSPYTEVQEFAIVPLHAAPTEAVSEIDALYDVYLDVWQKWGLEDIMFMGDEN
AGCSYVTSSQWSSIRLRTSPIFQWLIPDSADTTVTSTHCAYDRIVVAGALLQAAVVPNSAVPFD
FQAEYGLSNQLAEAISDHYPVEVTLRKISSTMVGSGCKPCICTVPEVSSVFIFPPKPKDVLYIT
LEPKVTCVVVDISKDDPEVQFSWFVDDVEVHTAQTQPREEQFNSTFRSVSELPIMHQDWLNGKE
FKCRVNSAAFPAPIEKTISKTKGRPKAPQVYTIPPPKEQMAKDKVSLTCMITDFFPEDITVEWQ
WNGQPAENYKNTQPIMDTDGSYFVYSKLNVQKSNWEAGNTFTCSVLHEGLHNHHTEKSLSHSPG
K
(Sequence 1689 - mouse DNAse1 construct)
SEQ ID NO: 35
MRYTGLMGTLLTLVNLLQLAGTLRIAAFNIRTFGRTKMSNATLSVYFVKILSRYDIAVIQEVRD
SHLVAVGKLLDELNRDKPDTYRYNVSEPLGRKSYKEQYLFVYRPDQVSILDSYQYDDGCEPCGN
DTFSREPAIVKFFSPYTEVQEFAIVPLHAAPTEAVSEIDALYDVYLDVWQKWGLEDIMFMGDEN
AGCSYVTSSQWSSIRLRTSPIFQWLIPDSADTTVTSTHCAYDRIVVAGALLQAAVVPNSAVPFD
FQAEYGLSNQTAEAISDHYPVEVTLRKISSTMVGSGCKPCICTVPEVSSVFIFPPKPKDVLYIT
LEPKVTCVVVDISKDDPEVQFSWFVDDVEVHTAQTQPREEQFNSTERSVSELPIMHQDWLNGKE
FKCRVNSAAFPAPIEKTISKTKGRPKAPQVYTIPPPKEQMAKDKVSLTCMITDFFPEDITVEWQ
WNGQPAENYKNTQPIMDTDGSYFVYSKLNVQKSNWEAGNTFTCSVLHEGLHNHHTEKSLSHSPG
K
(Sequence 1584 - mouse DNAse1L3 construct)
SEQ ID NO: 36
MSLHPASPRLASLLLFILALHDTLALRLCSFNVRSFGASKKENHEAMDIIVKIIKRCDLILLME
IKDSSNNICPMLMEKLNGNSRRSTTYNYVISSRLGRNTYKEQYAFVYKEKLVSVKTKYHYHDYQ
DGDTDVFSREPFVVWFHSPFTAVKDFVIVPLHTTPETSVKEIDELVDVYTDVRSQWKTENFIFM
GDFNAGCSYVPKKAWQNIRLRTDPKFVWLIGDQEDTTVKKSTSCAYDRIVLCGQEIVNSVVPRS
SGVFDFQKAYDLSEEEALDVSDHFPVEFKLQSSRAFTNNRKSVSLKKRKKGNRSSSTMVGSGCK
PCICTVPEVSSVFIFPPKPKDVLYITLEPKVTCVVVDISKDDPEVQFSWFVDDVEVHTAQTQPR
EEQFNSTFRSVSELPIMHQDWLNGKEFKCRVNSAAFPAPIEKTISKTKGRPKAPQVYTIPPPKE
QMAKDKVSLTCMITDFFPEDITVEWQWNGQPAENYKNTQPIMDTDGSYFVYSKLNVQKSNWEAG
NTFTCSVLHEGLHNHHTEKSLSHSPGK
(Sequence 1596 - mouse DNAse1L3 construct)
SEQ ID NO: 37
MSLHPASPRLASLLLFILALHDTLALRLCSFNVRSFGASKKENHEAMDIIVKIIKRCDLILLME
IKDSSNNICPMLMEKLNGNSRRSTTYNYVISSRLGRNTYKEQYAFVYKEKLVSVKTKYHYHDYQ
DGDTDVFSREPFVVWFHSPFTAVKDFVIVPLHTTPETSVKEIDELVDVYTDVRSQWKTENFIFM
GDFNAGCSYVPKKAWQNIRLRTDPKFVWLIGDQEDTTVKKSTSCAYDRIVLCGQEIVNSVVPRS
SGVFDFQKAYDLSEEEALDVSDHFPVEFKLQSSRAFTNNRSGCKPCICTVPEVSSVFIFPPKPK
DVLYITLEPKVTCVVVDISKDDPEVQFSWFVDDVEVHTAQTQPREEQENSTERSVSELPIMHQD
WLNGKEFKCRVNSAAFPAPIEKTISKTKGRPKAPQVYTIPPPKEQMAKDKVSLTCMITDFFPED
ITVEWQWNGQPAENYKNTQPIMDTDGSYFVYSKLNVQKSNWEAGNTFTCSVLHEGLHNHHTEKS
LSHSPGK
(Sequence 1615 - mouse DNAse1L3 construct)
SEQ ID NO: 38
MSLHPASPRLASLLLFILALHDTLALRLCSFNVRSFGRSKKENHEAMDIIVKIIKRCDLILLME
IKDSSNNICPMLMEKLNGNSRRSTTYNYVISSRLGRKTYKEQYAFVYKEKLVSVKTKYHYHDYQ
DGDTDVFSREPFVVWFHSPFTAVKDFVIVPLHTTPETSVKEIDELVDVYTDVRSQWKTENFIFM
GDFNAGCSYVPKKAWQNIRLRTDPKFVWLIGDQEDTTVKKSTSCAYDRIVLCGQEIVNSVVPRS
SGVFDFQKAYDLSEEEALDVSDHFPVEFKLQSSRAFTNNRKSVSLKKRKKGNRSSSTMVGSGCK
PCICTVPEVSSVFIFPPKPKDVLYITLEPKVTCVVVDISKDDPEVQFSWFVDDVEVHTAQTQPR
EEQFNSTFRSVSELPIMHQDWLNGKEFKCRVNSAAFPAPIEKTISKTKGRPKAPQVYTIPPPKE
QMAKDKVSLTCMITDFFPEDITVEWQWNGQPAENYKNTQPIMDTDGSYFVYSKLNVQKSNWEAG
NTFTCSVLHEGLHNHHTEKSLSHSPGK
(Sequence 1669 - mouse DNAse1L3 construct)
SEQ ID NO: 39
MSLHPASPRLASLLLFILALHDTLALRLCSFNVRSFGRSKKENHEAMDIIVKIIKRCDLILLME
IKDSSNNICPMLMEKLNGNSRRSTTYNYVISSRLGRKTYKEQYAFVYKEKLVSVKTKYHYHDYQ
DGDTDVFSREPFVVWFHSPFTAVKDFVIVPLHTTPETSVKEIDELVDVYTDVRSQWKTENFIFM
GDFNAGCSYVPKKAWQNIRLRTDPKFVWLIGDQEDTTVKKSTSCAYDRIVLCGQEIVNSVVPRS
NGTFDFQKAYDLSEEEALDVSDHFPVEFKLQSSRAFTNNRKSVSLKKRKKGNRSSSTMVGSGCK
PCICTVPEVSSVFIFPPKPKDVLYITLEPKVTCVVVDISKDDPEVQFSWFVDDVEVHTAQTQPR
EEQFNSTERSVSELPIMHQDWLNGKEFKCRVNSAAFPAPIEKTISKTKGRPKAPQVYTIPPPKE
QMAKDKVSLTCMITDFFPEDITVEWQWNGQPAENYKNTQPIMDTDGSYFVYSKLNVQKSNWEAG
NTFTCSVLHEGLHNHHTEKSISHSPGK

In certain embodiments, the present disclosure contemplates a construct that is expressed from a mammalian cell line, such as but not limited to a CHO cell line, which is stably transfected with human ST6 beta-galactosamide alpha-2,6-sialyltransferase (ST6GAL1). In certain embodiments, such expression enhances sialyation of the construct. The present disclosure further provides a construct that is grown in a cell culture supplemented with sialic acid and/or N-acetylmannosamine (1,3,4-O-Bu3ManNAc). In certain embodiments, such growth enhances sialic acid capping of the construct.

In certain embodiments, enhancing protein sialyation by expressing the biologic in CHO cells stably transfected with human alpha-2,6-sialyltransferase substantially improved construct bioavailability (Cₘₐₓ) when dosed subcutaneously. In other embodiments, increasing the pH-dependent FcRn-mediated cellular recycling by manipulating the Fc domain led to improvements of in vito biologic half-life. In yet other embodiments, combining CHO cells stably transfected with human α-2,6-sialyltransferase and growing the cells in N-acetylmannosamine led to dramatic increases half-life and/or biologic exposure (AUC). In yet other embodiments, combining two or more methods described herein into a single construct led to dramatic increases in half-life and/or biologic exposure (AUC).

In certain embodiments, the constructs of the disclosure are more highly glycosylated than other DNAse1 and/or DNAse1L3 constructs in the art. In other embodiments, the constructs of the disclosure have higher affinity for the neonatal orphan receptor (FcRn) than other DNAse1 and/or DNAse1L3 constructs in the art. In yet other embodiments, the constructs of the disclosure have higher in vito half-lives than other DNAse1 and/or DNAse1L3 constructs in the art. In yet other embodiments, the in vivo half-life of a construct of the disclosure is at least about 1.5, 2, 2.5, 3, 4, 5, 6, 7, 8, 9, 10, 12, 14, 16, 18, or 20 times higher than the DNAse1 and/or DNAse1L3 constructs described in the art. In yet other embodiments, the constructs of the disclosure are administered to the subject at a lower dose and/or at a lower frequency than other DNAse1 and/or DNAse1L3 constructs in the art. In yet other embodiments, the constructs of the disclosure are administered to the subject once a month, twice a month, three times a month, and/or four times a month. In yet other embodiments, the lower frequency administration of the constructs of the disclosure results in better patient compliance and/or increased efficacy as compared with other DNAse1 and/or DNAse1L3 constructs in the art.

In certain embodiments, the construct is soluble. In other embodiments, the construct is a recombinant polypeptide.

In certain embodiments, the construct comprises a signal peptide resulting in the secretion of a precursor of the DNAse1 and/or DNAse1L3 polypeptide, which undergoes proteolytic processing to yield a processed construct comprising the DNAse1 and/or DNAse1L3 polypeptide.

In certain embodiments, the DNAse1 and/or DNAse1L3 polypeptide is C-terminally fused to the Fc domain of human immunoglobulin 1 (IgG1), human immunoglobulin 2 (IgG2), human immunoglobulin 3 (IgG3), and/or human immunoglobulin 4 (IgG4). In other embodiments, the DNAse1 and/or DNAse1L3 polypeptide is N-terminally fused to the Fc domain of human immunoglobulin 1 (IgG1), human immunoglobulin 2 (IgG2), human immunoglobulin 3 (IgG3), and/or human immunoglobulin 4 (IgG4). In yet other embodiments, the presence of IgFc domain improves half-life, solubility, reduces immunogenicity, and increases the activity of the DNAse1 and/or DNAseL3 polypeptide.

In certain embodiments, the DNAse1 and/or DNAse1L3 polypeptide is C-terminally fused to human serum albumin. Human serum albumin may be conjugated to DNAse1 and/or DNAse1L3 protein through a chemical linker, including but not limited to naturally occurring or engineered disulfide bonds, and/or by genetic fusion to DNAse1 and/or DNAse1L3, and/or a fragment and/or variant thereof.

In certain embodiments, the construct is further pegylated (i.e., fused with a poly(ethylene glycol) chain).

In certain embodiments, the construct is formulated as a liquid formulation. In other embodiments, the disclosure provides a dry product form of a pharmaceutical composition comprising a therapeutic amount of a construct of the disclosure, whereby the dry product is reconstitutable to a solution of the construct in liquid form.

The disclosure provides a kit comprising at least one construct of the disclosure, and/or a salt or solvate thereof, and instructions for using the construct within the methods of the disclosure.

It will be understood that a DNAse1 and/or DNAse1 L3 polypeptide according to the disclosure includes not only the native human proteins, but also any fragment, derivative, fusion, conjugate or mutant thereof. As used herein in this disclosure, the phrase “a DNAse1 and/or DNAse1L3 polypeptide, mutant, and/or mutant fragment thereof” also includes any compound or polypeptide (such as, but not limited to, a fusion protein) comprising a DNAse1 and/or DNAse1 L3 polypeptide, mutant, and/or mutant fragment thereof. Fusion proteins according to the disclosure are considered biological equivalents of DNAse1 and/or DNAse1L3, but can in certain embodiments provide longer half-life or greater potency due to increased in vivo biologic exposure, as judged by the “area under the curve” (AUC) or increased half-life in pharmacokinetic experiments.

### Vectors and Cells

The disclosure further provides an autonomously replicating or an integrative mammalian cell vector comprising a recombinant nucleic acid encoding a polypeptide of the disclosure. In certain embodiments, the vector comprises a plasmid or a virus. In other embodiments, the vector comprises a mammalian cell expression vector. In yet other embodiments, the vector further comprises at least one nucleic acid sequence that directs and/or controls expression of the polypeptide. In yet other embodiments, the recombinant nucleic acid encodes a construct comprising a DNAse1 and/or DNAse1L3 polypeptide and a signal peptide, wherein the polypeptide is proteolytically processed upon secretion from a cell to yield the DNAse1 and/or DNAse1L3 construct of the disclosure.

In yet another aspect, the disclosure provides an isolated host cell comprising a vector of the disclosure. In certain embodiments, the cell is a non-human cell. In other embodiments, the cell is mammalian. In yet other embodiments, the vector of the disclosure comprises a recombinant nucleic acid encoding a construct comprising a DNAse1 and/or DNAse1L3 polypeptide and a signal peptide. In yet other embodiments, the polypeptide is proteolytically processed upon secretion from a cell to yield the DNAse1 and/or DNAse1L3 construct of the disclosure.

Production and Purification of DNAse1 and/or DNAse1L3 Fusion Proteins

In certain embodiments, a soluble DNAse1 and/or DNAse1L3 construct, including IgG Fc domain or enzymatically/biologically active fragments thereof, are efficacious in treating, reducing, and/or preventing progression of diseases or disorders contemplated herein.

To produce soluble, recombinant DNAse1 and/or DNAse1 L3 constructs for in vitro use, DNAse1 and/or DNAse1L3 polypeptides can be fused to the Fc domain of IgG (referred to as “DNAse1-Fc” or “DNAse1 L3-Fc”) and the fusion construct can be expressed in stable CHO cell lines. The construct can also be expressed from HEK293 cells, Baculovirus insect cell system or CHO cells or Yeast Pichia expression system using suitable vectors. The construct can be produced in either adherent or suspension cells. To establish stable cell lines the nucleic acid sequence encoding DNAse1 and/or DNAseL3 constructs are cloned into an appropriate vector for large scale protein production.

Many expression systems are known can be used for the production of DNAse1 and/or DNAse1L3 constructs, including bacteria (for example E. coli and Bacillus subtilis), yeasts (for example Saccharomyces cerevisiae, Kluyveronmyces laclis and Pichia pastoris), filamentous fungi (for example Aspergillus), plant cells, animal cells and insect cells. The desired proteins can be produced in conventional ways, for example from a coding sequence inserted in the host chromosome or on a free plasmid.

The yeasts can be transformed with a coding sequence for the desired protein in any one of the usual ways, for example electroporation. Methods for transformation of yeast by electroporation are disclosed in Becker & Guarente, 1990, Methods Enzymol. 194: 182. Successfully transformed cells, i.e., cells that contain a DNA construct of the present disclosure, can be identified by well-known techniques. For example, cells resulting from the introduction of an expression construct can be grown to produce the desired polypeptide. Cells can be harvested and lysed and their DNA content examined for the presence of the DNA using a method, such as that described by Southern, 1975, J. Mol. Biol, 98:503 and/or Berent, et al., 1985, Biotech 3:208. Alternatively, the presence of the protein in the supernatant can be detected using antibodies.

Useful yeast plasmid vectors include pRS403-406 and pRS413-416 and are generally available fron1 Strat:1.gene Cloning Systems, La Jolla, CA, USA Plasmids pRS403, pRS404, pRS405 and pRS406 are Yeast Integrating plasmids (Ylps) and incorporate the yeast selectable markers I-llS3, TRP1, LEU2 and 1JRA3. Plasmids pRS413-416 are Yeast Centromere plasmids (YCps).

A variety of methods have been developed to operably link DNA to vectors via complementary cohesive termini. For instance, complementary homopolymer tract can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase I, which are enzymes that remove protruding, 3′-single-stranded termini with their 3′-5′-exonucleolytic activities, and fill in recessed 3′-ends with their polymerizing activities.

The combination of these activities thus generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Clones of single, stably transfected cells are then established and screened for high expressing clones of the desired fusion protein. Screening of the single cell clones for DNAse1 and/or DNAse1L3 protein expression can be accomplished in a high-throughput manner in 96 well plates. Upon identification of high expressing clones through screening, protein production can be accomplished in shaking flasks or bio-reactors.

Purification of DNAse1 and/or DNAse1L3 constructs can be accomplished using a combination of standard purification techniques known in the art.

### Gene Therapy

The nucleic acids encoding the polypeptide(s) useful within the disclosure may be used in gene therapy protocols for the treatment of the diseases or disorders contemplated herein. The improved construct encoding the polypeptide(s) can be inserted into the appropriate gene therapy vector and administered to a patient to treat or prevent the diseases or disorder of interest.

Vectors, such as viral vectors, have been used in the prior art to introduce genes into a wide variety of different target cells. Typically the vectors are exposed to the target cells so that transformation can take place in a sufficient proportion of the cells to provide a useful therapeutic or prophylactic effect from the expression of the desired polypeptide (e.g., a receptor). The transfected nucleic acid may be permanently incorporated into the genome of each of the targeted cells, providing long lasting effect, or alternatively the treatment may have to be repeated periodically. In certain embodiments, the (viral) vector transfects liver cells in vivo with genetic material encoding the polypeptide(s) of the disclosure.

A variety of vectors, both viral vectors and plasmid vectors are known in the art (see for example U.S. Pat. No. 5,252,479 and WO 93/07282). In particular, a number of viruses have been used as gene transfer vectors, including papovaviruses, such as SV40, vaccinia virus, herpes viruses including HSV and EBV, and retroviruses. Many gene therapy protocols in the prior art have employed disabled murine retroviruses. Several recently issued patents are directed to methods and compositions for performing gene therapy (see for example U.S. Pat. Nos. 6,168,916; 6,135,976; 5,965,541 and 6,129,705). Each of the foregoing patents is incorporated by reference in its entirety herein.

### AAV-Mediated Gene Therapy:

AAV, a parvovirus belonging to the genus Dependovirus, has several features that make it particularly well suited for gene therapy applications. For example, AAV can infect a wide range of host cells, including non-dividing cells. Furthermore, AAV can infect cells from a variety of species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, which is a linear, single-stranded DNA molecule containing approximately 4,700 nucleotides (the AAV-2 genome consists of 4,681 nucleotides, the AAV-4 genome 4,767), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, which allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, that is, it requires co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV replicates in canine cells that have been co-infected with a canine adenovirus.

To produce infectious recombinant AAV (rAAV) containing a heterologous nucleic acid sequence, a suitable host cell line can be transfected with an AAV vector containing the heterologous nucleic acid sequence, but lacking the AAV helper function genes, rep and cap. The AAV-helper function genes can then be provided on a separate vector. Also, only the helper virus genes necessary for AAV production (i.e., the accessory function genes) can be provided on a vector, rather than providing a replication-competent helper virus (such as adenovirus, herpesvirus, or vaccinia).

Collectively, the AAV helper function genes (i.e., rep and cap) and accessory function genes can be provided on one or more vectors. Helper and accessory function gene products can then be expressed in the host cell where they will act in trans on rAAV vectors containing the heterologous nucleic acid sequence. The rAAV vector containing the heterologous nucleic acid sequence will then be replicated and packaged as though it were a wild-type (wt) AAV genome, forming a recombinant virion. When a patient's cells are infected with the resulting rAAV virions, the heterologous nucleic acid sequence enters and is expressed in the patient's cells. Because the patient's cells lack the rep and cap genes, as well as the accessory function genes, the rAAV cannot further replicate and package their genomes. Moreover, without a source of rep and cap genes, wtAAV cannot be formed in the patient's cells.

There are eleven known AAV serotypes, AAV-1 through AAV-11 (Mori, et al., 2004, Virology 330(2):375-83). AAV-2 is the most prevalent serotype in human populations; one study estimated that at least 80% of the general population has been infected with wt AAV-2 (Berns and Linden, 1995, Bioessays 17:237-245). AAV-3 and AAV-5 are also prevalent in human populations, with infection rates of up to 60% (Georg-Fries, et al., 1984, Virology 134:64-71). AAV-1 and AAV-4 are simian isolates, although both serotypes can transduce human cells (Chiorini, et al., 1997, J Virol 71:6823-6833; Chou, et al., 2000, Mol Ther 2:619-623). Of the six known serotypes, AAV-2 is the best characterized. For instance, AAV-2 has been used in a broad array of in vivo transduction experiments, and has been shown to transduce many different tissue types including: mouse (U.S. Pat. Nos. 5,858,351; 6,093,392), dog muscle; mouse liver (Couto, et al., 1999, Proc. Natl. Acad. Sci. USA 96:12725-12730; Couto, et al., 1997, J. Virol. 73:5438-5447; Nakai, et al., 1999, J. Virol. 73:5438-5447; and, Snyder, et al., 1997, Nat. Genet. 16:270-276): mouse heart (Su, et al., 2000, Proc. Natl. Acad. Sci. USA 97:13801-13806); rabbit lung (Flotte, et al., 1993, Proc. Natl. Acad. Sci. USA 90:10613-10617); and rodent photoreceptors (Flannery et al., 1997, Proc. Natl. Acad. Sci. USA 94:6916-6921).

The broad tissue tropism of AAV-2 may be exploited to deliver tissue-specific transgenes. For example, AAV-2 vectors have been used to deliver the following genes: the cystic fibrosis transmembrane conductance regulator gene to rabbit lungs (Flotte, et al., 1993, Proc. Natl. Acad. Sci. USA 90:10613-10617); Factor NIII gene (Burton, et al., 1999, Proc. Natl. Acad. Sci. USA 96:12725-12730) and Factor IX gene (Nakai, et al., 1999, J. Virol. 73:5438-5447; Snyder, et al., 1997, Nat. Genet. 16:270-276; U.S. Pat. No. 6,093,392) to mouse liver, dog, and mouse muscle (U.S. Pat. No. 6,093,392); erythropoietin gene to mouse muscle (U.S. Pat. Nos. 5,858,351); vascular endothelial growth factor (VEGF) gene to mouse heart (Su, et al., 2000, Proc. Natl. Acad. Sci. USA 97:13801-13806); and aromatic 1-amino acid decarboxylase gene to monkey neurons. Expression of certain rAAV-delivered transgenes has therapeutic effect in laboratory animals; for example, expression of Factor IX was reported to have restored phenotypic normalcy in dog models of hemophilia B (U.S. Pat. No. 6,093,392). Moreover, expression of rAAV-delivered NEGF to mouse myocardium resulted in neovascular formation (Su, et al., 2000, Proc. Natl. Acad. Sci. USA 97:13801-13806), and expression of rAAV-delivered AADC to the brains of parkinsonian monkeys resulted in the restoration of dopaminergic function.

Delivery of a protein of interest to the cells of a mammal is accomplished by first generating an AAV vector comprising DNA encoding the protein of interest and then administering the vector to the mammal. Thus, the disclosure should be construed to include AAV vectors comprising DNA encoding the polypeptide(s) of interest. Once armed with the present disclosure, the generation of AAV vectors comprising DNA encoding this/these polypeptide(s)s will be apparent to the skilled artisan.

In certain embodiments, the rAAV vector of the disclosure comprises several essential DNA elements. In certain embodiments, these DNA elements include at least two copies of an AAV ITR sequence, a promoter/enhancer element, a transcription termination signal, any necessary 5′ or 3′ untranslated regions which flank DNA encoding the protein of interest or a biologically active fragment thereof. The rAAV vector of the disclosure may also include a portion of an intron of the protein on interest. Also, optionally, the rAAV vector of the disclosure comprises DNA encoding a mutated polypeptide of interest.

In certain embodiments, the vector comprises a promoter/regulatory sequence that comprises a promiscuous promoter which is capable of driving expression of a heterologous gene to high levels in many different cell types. Such promoters include, but are not limited to the cytomegalovirus (CMV) immediate early promoter/enhancer sequences, the Rous sarcoma virus promoter/enhancer sequences and the like. In certain embodiments, the promoter/regulatory sequence in the rAAV vector of the disclosure is the CMV immediate early promoter/enhancer. However, the promoter sequence used to drive expression of the heterologous gene may also be an inducible promoter, for example, but not limited to, a steroid inducible promoter, or may be a tissue specific promoter, such as, but not limited to, the skeletal a-actin promoter which is muscle tissue specific and the muscle creatine kinase promoter/enhancer, and the like.

In certain embodiments, the rAAV vector of the disclosure comprises a transcription termination signal. While any transcription termination signal may be included in the vector of the disclosure, in certain embodiments, the transcription termination signal is the SV40 transcription termination signal.

In certain embodiments, the rAAV vector of the disclosure comprises isolated DNA encoding the polypeptide of interest, or a biologically active fragment of the polypeptide of interest. The disclosure should be construed to include any mammalian sequence of the polypeptide of interest, which is either known or unknown. Thus, the disclosure should be construed to include genes from mammals other than humans, which polypeptide functions in a substantially similar manner to the human polypeptide. Preferably, the nucleotide sequence comprising the gene encoding the polypeptide of interest is about 50% homologous, more preferably about 70% homologous, even more preferably about 80% homologous and most preferably about 90% homologous to the gene encoding the polypeptide of interest.

Further, the disclosure should be construed to include naturally occurring variants or recombinantly derived mutants of wild type protein sequences, which variants or mutants render the polypeptide encoded thereby either as therapeutically effective as full-length polypeptide, or even more therapeutically effective than full-length polypeptide in the gene therapy methods of the disclosure.

The disclosure should also be construed to include DNA encoding variants which retain the polypeptide's biological activity. Such variants include proteins or polypeptides which have been or may be modified using recombinant DNA technology, such that the protein or polypeptide possesses additional properties which enhance its suitability for use in the methods described herein, for example, but not limited to, variants conferring enhanced stability on the protein in plasma and enhanced specific activity of the protein. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function.

The disclosure is not limited to the specific rAAV vector exemplified in the experimental examples; rather, the disclosure should be construed to include any suitable AAV vector, including, but not limited to, vectors based on AAV-1, AAV-3, AAV-4 and AAV-6, and the like.

Also included in the disclosure is a method of treating a mammal having a disease or disorder in an amount effective to provide a therapeutic effect. The method comprises administering to the mammal an rAAV vector encoding the polypeptide of interest. Preferably, the mammal is a human.

Typically, the number of viral vector genomes/mammal which are administered in a single injection ranges from about 1×10⁸ to about 5×10¹⁶. Preferably, the number of viral vector genomes/mammal which are administered in a single injection is from about 1×10¹⁰ to about 1×10¹⁵; more preferably, the number of viral vector genomes/mammal which are administered in a single injection is from about 5-10¹⁰ to about 5×10¹⁵; and, most preferably, the number of viral vector genomes which are administered to the mammal in a single injection is from about 5×10¹¹ to about 5-10¹⁴.

When the method of the disclosure comprises multiple site simultaneous injections, or several multiple site injections comprising injections into different sites over a period of several hours (for example, from about less than one hour to about two or three hours) the total number of viral vector genomes administered may be identical, or a fraction thereof or a multiple thereof, to that recited in the single site injection method.

For administration of the rAAV vector of the disclosure in a single site injection, in certain embodiments a composition comprising the virus is injected directly into an organ of the subject (such as, but not limited to, the liver of the subject).

For administration to the mammal, the rAAV vector may be suspended in a pharmaceutically acceptable carrier, for example, HEPES buffered saline at a pH of about 7.8. Other useful pharmaceutically acceptable carriers include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The rAAV vector of the disclosure may also be provided in the form of a kit, the kit comprising, for example, a freeze-dried preparation of vector in a dried salts formulation, sterile water for suspension of the vector/salts composition and instructions for suspension of the vector and administration of the same to the mammal.

### Methods

The disclosure includes a method of treating, ameliorating, and/or preventing forms of lupus (including SLE) associated with DNAse1L3 deficiency.

The disclosure includes a method of treating, ameliorating, and/or preventing diseases and/or disorders associated with inefficient NET hydrolysis (“NETolysis”).

The disclosure includes a method of treating, ameliorating, and/or preventing autoimmune disorders. In certain embodiments, the autoimmune disorders comprise lupus (including SLE), thyroid autoimmune disease, and/or Hypocomplementeric Urticarial Vasculitis Syndrome (HUVS).

The disclosure includes a method of treating, ameliorating, and/or preventing pathologic thrombosis, such as but not limited to microvascular thrombosis, venous thrombosis, and/or arterial thrombosis. In certain embodiments, the pathologic thrombosis comprises neutrophilic thrombosis, which includes but is not limited to Anti-Neutrophilic Cytoplasmic Autoantibodies (ANCA) vasculitis, Thrombotic thrombocytopenic purpura (TTP), and Bechet's (or Behcet's) disease or syndrome. In certain embodiments, the pathologic thrombosis comprises thrombosis leading to strokes.

The disclosure includes a method of treating, ameliorating, and/or preventing myocardial infarctions.

The disclosure includes a method of treating, ameliorating, and/or preventing spread and progression of cancer (e.g., cancer metastasis).

In certain embodiments, the method comprises administering a construct of the disclosure to the subject who is suffering from, suspect of suffering from, and/or likely to develop any disease or disorder contemplated herein.

In certain embodiments, the construct of the disclosure is a secreted product of a DNAse1 and/or DNAse1 L3 precursor construct (which is itself a construct contemplated within the disclosure) expressed in a mammalian cell. In other embodiments, the DNAse1 and/or DNAse1 L3 precursor construct comprises a signal peptide sequence and a DNAse1 and/or DNAse1 L3 polypeptide, wherein the DNAse1 and/or DNAse1 L3 precursor construct undergoes proteolytic processing to a processed construct comprising the DNAse1 and/or DNAse1 L3 polypeptide. In yet other embodiments, in the DNAse1 and/or DNAse1 L3 precursor construct the signal peptide sequence is conjugated to the DNAse1 and/or DNAse1 L3 polypeptide N-terminus. Upon proteolysis, the signal sequence is cleaved from the DNAse1 and/or DNAse1 L3 precursor construct to provide the construct comprising the DNAse1 and/or DNAse1 L3 polypeptide.

In certain embodiments, the construct is administered acutely or chronically to the subject. In other embodiments, the construct is administered locally, regionally, parenterally or systemically to the subject

In certain embodiments, the subject is a mammal. In other embodiments, the mammal is human.

In certain embodiments, the construct, and/or its precursor construct, is administered by at least one route selected from the group consisting of subcutaneous, oral, aerosol, inhalational, rectal, vaginal, transdermal, subcutaneous, intranasal, buccal, sublingual, parenteral, intrathecal, intragastrical, ophthalmic, pulmonary and topical. In other embodiments, the construct, and/or its precursor construct, is administered to the subject as a pharmaceutical composition further comprising at least one pharmaceutically acceptable carrier.

In certain embodiments, the construct, and/or its precursor construct, is administered acutely or chronically to the subject. In other embodiments, the construct, and/or its precursor construct, is administered locally, regionally or systemically to the subject. In yet another embodiment, the construct, and/or its precursor construct, is delivered on an encoded vector, wherein the vector encodes the protein and it is transcribed and translated from the vector upon administration of the vector to the subject.

It will be appreciated by one of skill in the art, when armed with the present disclosure including the methods detailed herein, that the disclosure is not limited to treatment of a disease or disorder once it is established. Particularly, the symptoms of the disease or disorder need not have manifested to the point of detriment to the subject; indeed, the disease or disorder need not be detected in a subject before treatment is administered. That is, significant pathology from disease or disorder does not have to occur before the present disclosure may provide benefit.

Thus, the present disclosure, as described more fully herein, includes a method for preventing diseases and disorders in a subject, in that a polypeptide or construct of the disclosure, as discussed elsewhere herein, can be administered to a subject prior to the onset of the disease or disorder, thereby preventing the disease or disorder from developing. Particularly, where the symptoms of the disease or disorder have not manifested to the point of detriment to the subject; indeed, the disease or disorder need not be detected in a subject before treatment is administered. That is, significant pathology from the disease or disorder does not have to occur before the present disclosure may provide benefit. Therefore, the present disclosure includes methods for preventing or delaying onset, and/or reducing progression or growth, of a disease or disorder in a subject, in that a polypeptide of the disclosure can be administered to a subject prior to detection of the disease or disorder. In certain embodiments, the polypeptide of the disclosure is administered to a subject with a strong family history of the disease or disorder, thereby preventing or delaying onset or progression of the disease or disorder.

Armed with the disclosure herein, one skilled in the art would thus appreciate that the prevention of a disease or disorder in a subject encompasses administering to a subject a polypeptide of the disclosure as a preventative measure against the disease or disorder.

### Pharmaceutical Compositions and Formulations

The disclosure provides pharmaceutical compositions comprising a polypeptide of the disclosure within the methods described herein.

Such a pharmaceutical composition is in a form suitable for administration to a subject, and/or the pharmaceutical composition may further comprise one or more pharmaceutically acceptable carriers, one or more additional ingredients, and/or some combination of these. The various components of the pharmaceutical composition may be present in the form of a physiologically acceptable salt, such as in combination with a physiologically acceptable cation or anion, as is well known in the art.

In an embodiment, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 500 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between about 0.1% and about 100% (w/w) active ingredient.

Pharmaceutical compositions that are useful in the methods of the disclosure may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

### Administration/Dosing

The regimen of administration may affect what constitutes an effective amount. For example, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder in the patient. An effective amount of the therapeutic compound necessary to achieve a therapeutic effect may vary according to factors such as the activity of the particular compound employed: the time of administration: the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. Dosage is determined based on the biological activity of the therapeutic compound which in turn depends on the half-life and the area under the plasma time of the therapeutic compound curve. The polypeptide according to the disclosure can be administered at an appropriate time interval of every 2 days, or every 4 days, or every week or every month. Therapeutic dosage of the polypeptides of the disclosure may also be determined based on half-life or the rate at which the therapeutic polypeptide is cleared out of the body. The polypeptide according to the disclosure is administered at appropriate time intervals of either every 2 days, or every 4 days, every week or every month so as to achieve a constant level of enzymatic activity of DNAse1 and/or DNAse1 L3.

For example, several divided doses may be administered daily or the dose may be proportionally reduced as indicated by the exigencies of the therapeutic situation. A non-limiting example of an effective dose range for a therapeutic compound of the disclosure is from about 0.01 and 50 mg/kg of body weight/per day. In some embodiments, the effective dose range for a therapeutic compound of the disclosure is from about 50 ng to 500 ng/kg, preferably 100 ng to 300 ng/kg of bodyweight. One of ordinary skill in the art would be able to study the relevant factors and make the determination regarding the effective amount of the therapeutic compound without undue experimentation.

The compound can be administered to a patient as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the patient.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. The frequency of administration of the various combination compositions of the disclosure varies from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physical taking all other factors about the patient into account.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

### Routes of Administration

Routes of administration of any one of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. The formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the disclosure may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Single unit dosage forms suitable for oral administration, such as tablets, capsules, gelcaps, and caplets, which are adapted for controlled-release are encompassed by the present disclosure.

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form. For sustained release, the compounds may be formulated with a suitable polymer or hydrophobic material that provides sustained release properties to the compounds. As such, the compounds for use the method of the disclosure may be administered in the form of microparticles, for example, by injection or in the form of wafers or discs by implantation. In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours. The term pulsatile release is used herein in its conventional sense to refer to a drug formulation that provides release of the drug in such a way as to produce pulsed plasma profiles of the drug after drug administration. The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction and preparation conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Methods and Materials

Unless specifically mentioned, expression of constructs in CHO cells or modified CHO cells with and without supplementation, enzymatic assays, AUC assay, half-life assay can be carried out using protocols described elsewhere herein or as known in the prior art.

### Area Under the Curve Assay

The area under the plasma concentration versus time curve, also called the area under the curve (AUC) can be used as a means of evaluating the volume of distribution (V), total elimination clearance (CL), and bioavailability (F) for extravascular drug delivery. Area under plasma time curve for each expressed and purified DNAse1-Fc and/or DNAse1L3-Fc construct can be carried out using the standard equation to determine half-life and bioavailability after a single subcutaneous injection of biologic, as described in Equation 1.

### Half-Life Determination

The drug half-life (t_{1/2}) is the time it takes for the plasma concentration or the amount of drug or biologic in the body to be reduced by 50%. Half-life values for each expressed and purified construct can be carried out following protocols described in the prior art and/or herein, such as Equation 1, which allows for determining half-life and bioavailability after a single subcutaneous injection of biologic.

Drug half-life can be calculated using Equation 1, which correlates the relationship between systemic fractional concentration and time of a drug administered to a subcutaneous depot in a single injection. Plotting the data as fraction of drug absorbed (F) over time (t) allows for the determination of the elimination (kₑ) and absorption (kₐ) constants by fitting the data to the equation for the total systemic absorption of a drug administered at a subcutaneous depot at time t=0.

$$F = \frac{{k}_{a}}{( {k}_{a} - {k}_{e} )} [ {e}^{- {k}_{e} t} - {e}^{- {k}_{a} t} ] \qquad ( Equation 1 )$$

### Examples

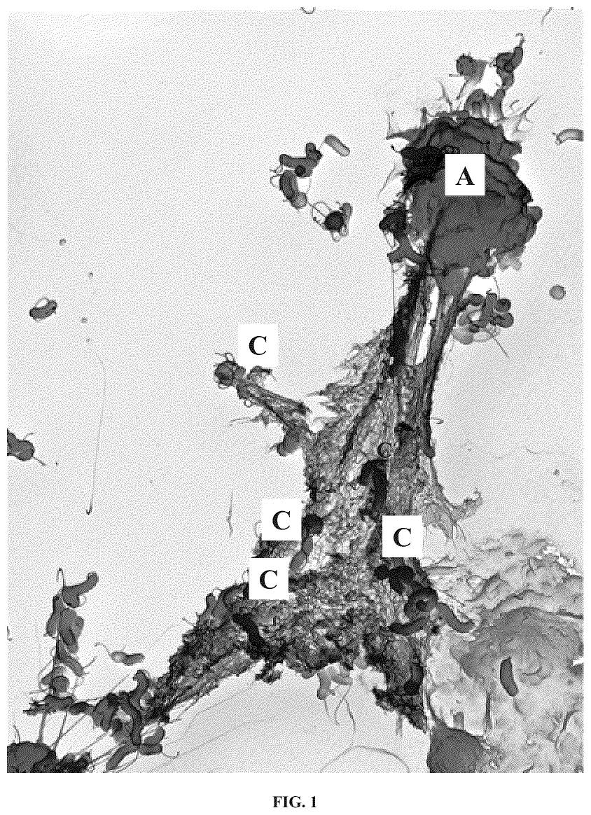

FIG. 1 illustrates neutrophil extracellular trap (NET) formation. Scanning electron microscopy of neutrophil (marked as A) casting a net (marked as B) entrapping Helicobacter pylori bacteria (some of which are marked as C). Image taken from Kumamoto T, et al., 2006, Eur Heart J. 27(17):2081-7.

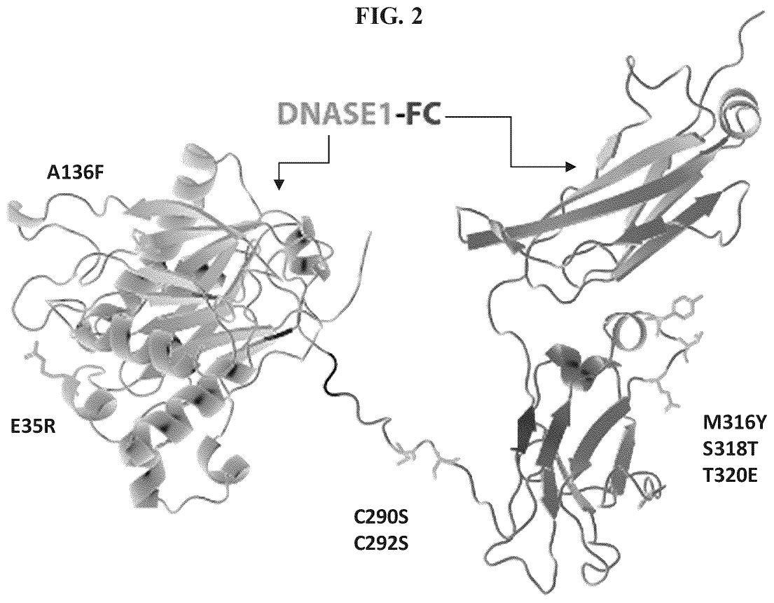

FIG. 2 illustrates a non-limiting DNAse1-Fc construct of the disclosure, with certain contemplated point mutations highlighted.

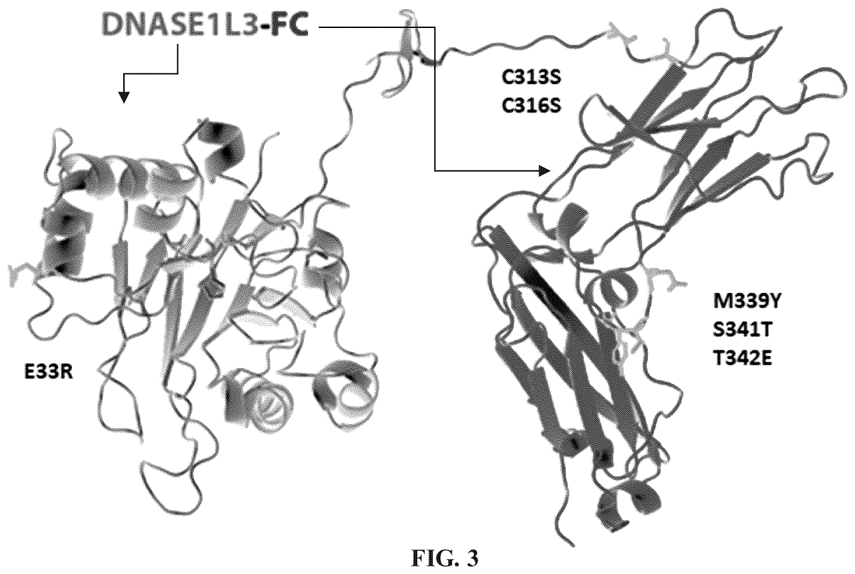

FIG. 3 illustrates a non-limiting DNAse1L3-Fc construct of the disclosure, with certain contemplated point mutations highlighted.

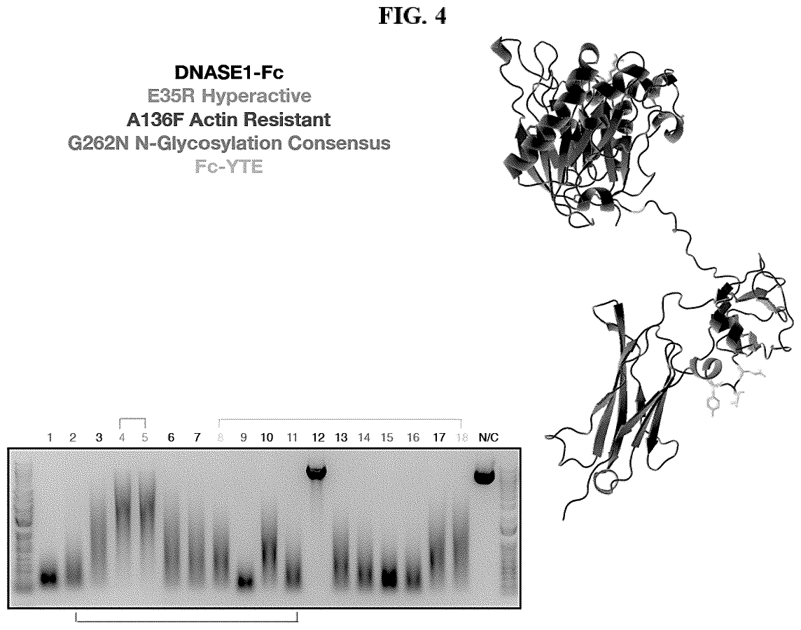

FIG. 4 illustrates a non-limiting DNAse1-Fc construct of the disclosure, with certain contemplated point mutations highlighted.

FIG. 5 illustrates non-limiting constructs of the disclosure, with certain contemplated point mutations highlighted. In certain embodiments, certain mutations render the rDNAse hyperactive and/or render the rDNAse actin-resistant (i.e., has decreased affinity for actin) and/or increase the construct's half-life.

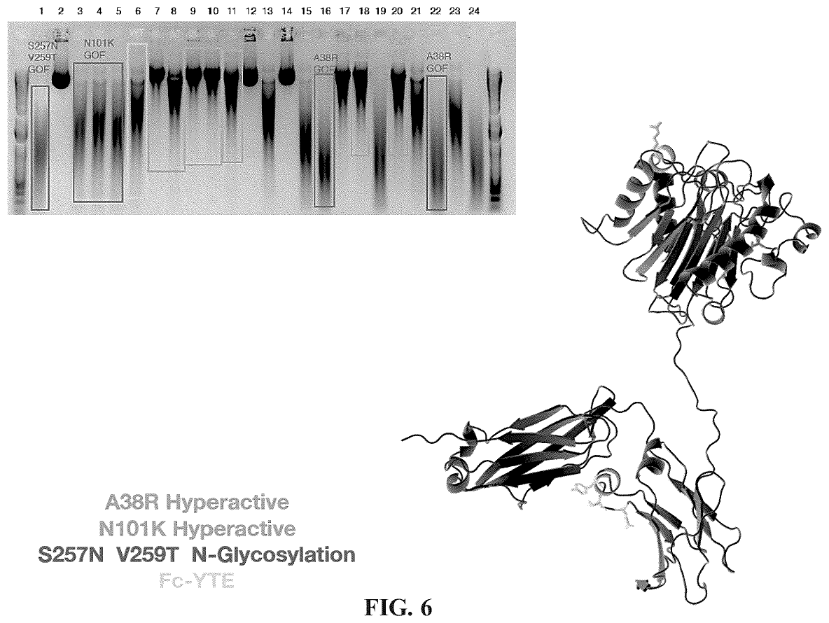

FIG. 6 illustrates non-limiting constructs of the disclosure, with certain contemplated point mutations highlighted. In certain embodiments, the construct lacks at least a portion of the DNAse1 L3 nuclear localization domain.

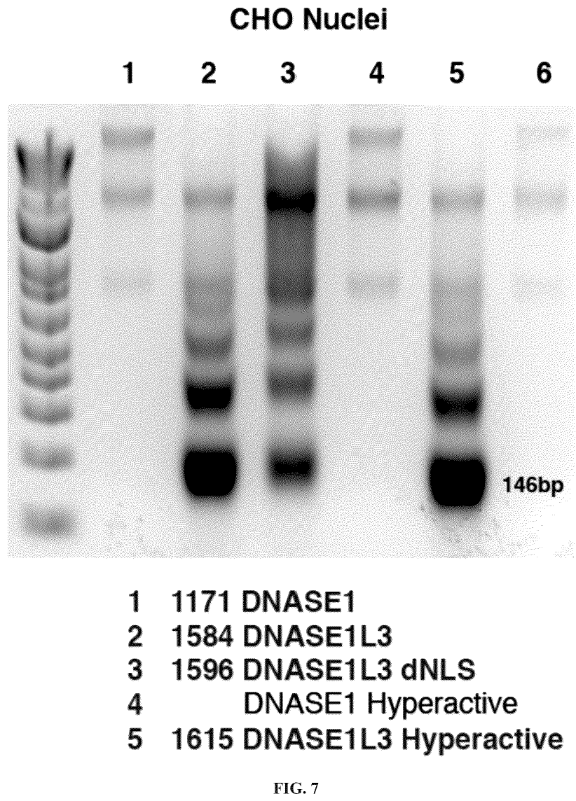

FIG. 7 illustrates a gel indicating that certain DNAse1 L3 clones cleave chromatin, but that is not the case for certain DNAse1 clones.

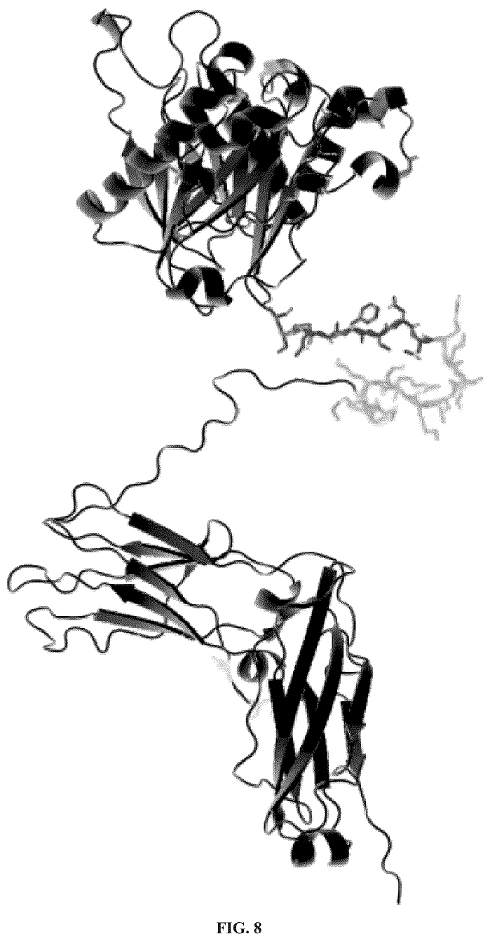

FIG. 8 illustrates a non-limiting construct of the disclosure. In certain embodiments, the DNAse1 polypeptide is fused with the C-terminus tail of DNAse1 L3.

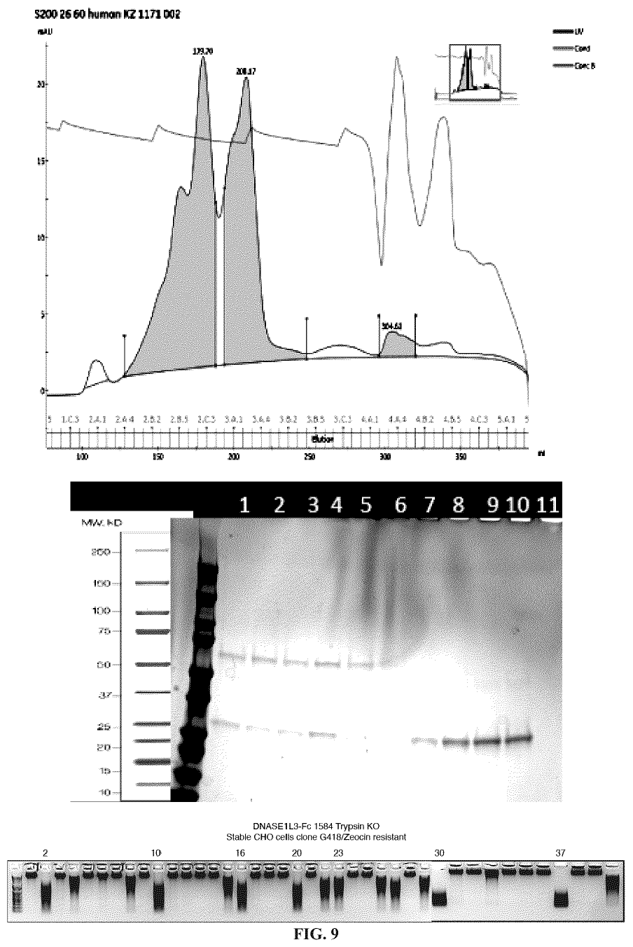

FIG. 9 illustrates certain aspects of production and purification of DNAse-Fc constructs.

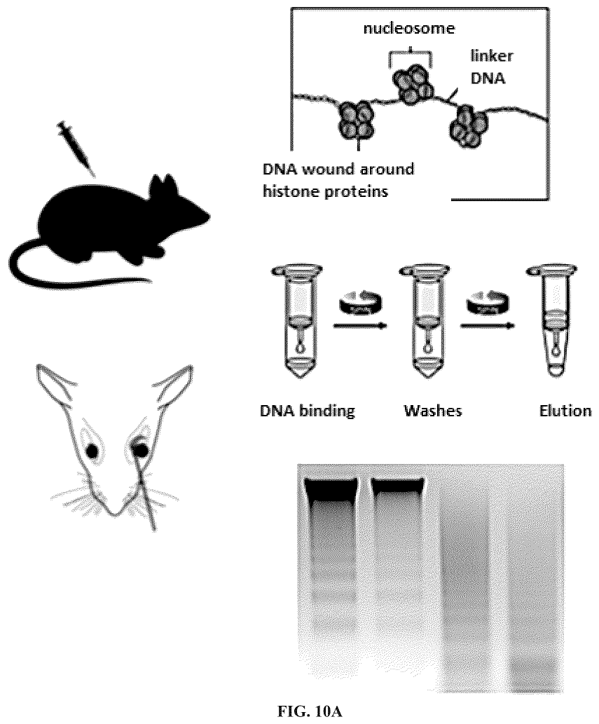

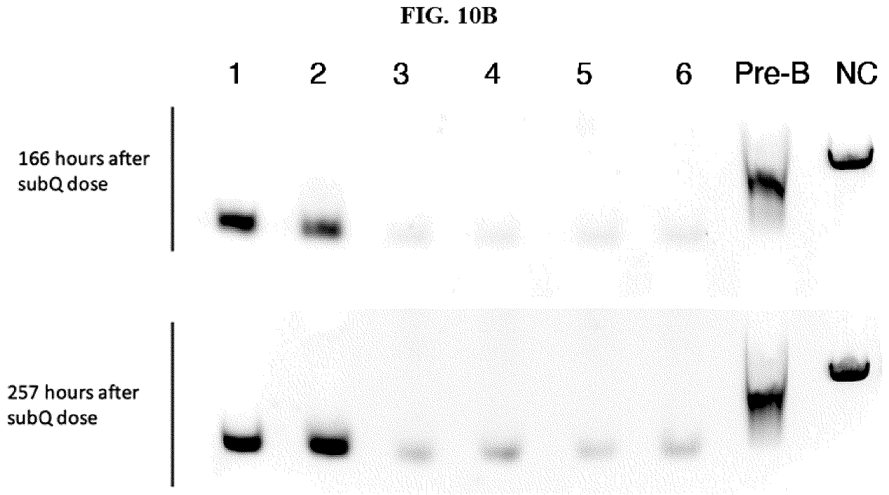

FIGS. 10A-10B illustrate in vivo pharmacodynamics of certain NET degrading constructs of the disclosure.

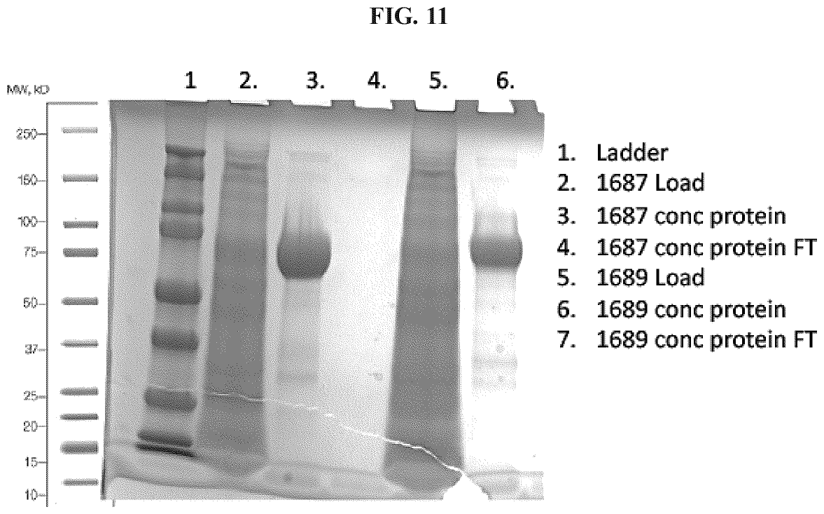

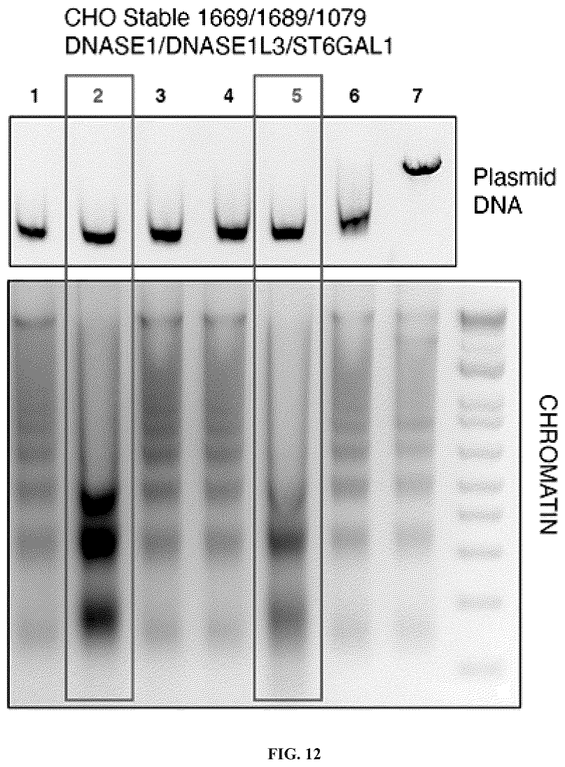

FIG. 11 illustrates a non-limiting purification gel of certain NET degrading constructs of the disclosure.

### Enumerated Embodiments

The following exemplary embodiments are provided, the numbering of which is not to be construed as designating levels of importance.

Embodiment 1 provides a construct comprising the amino acid sequence:

DNAse1-X1-LINKER-Fc-X2 (I)

wherein:
DNAse1 is a human DNAse1 polypeptide;
X1 is a covalent bond, or X1 is the peptide of amino acid sequence RAFTNNRKSVSLKKRKKGNRS (SEQ ID NO:3) or a fragment thereof;
LINKER is a chemical bond or a polypeptide comprising 1-100 amino acids;
X2 is null, or X2 is the peptide of amino acid sequence SEQ ID NO:3 or a fragment thereof;
Fc is the Fc domain of human IgG1.

Embodiment 1 provides a construct comprising the amino acid sequence:

DNAse1 L3-X1-LINKER-Fc-X2 (II)

wherein:
DNAse1 L3 is a human DNAse1 L3 polypeptide;
X1 is a covalent bond, or X1 is the peptide of amino acid sequence RAFTNNRKSVSLKKRKKGNRS (SEQ ID NO:3) or a fragment thereof;
LINKER is a covalent bond or a polypeptide comprising 1-100 amino acids;
X2 is null, or X2 is the peptide of amino acid sequence SEQ ID NO:3 or a fragment thereof;
Fc is the Fc domain of human IgG1.

Embodiment 3 provides the construct of any one of Embodiments 1-2, wherein the Fc comprises the amino acid sequence of SEQ ID NO:4.

Embodiment 4 provides the construct of Embodiment 3, wherein at least one of C6 and C9 with respect to SEQ ID NO:4 is independently mutated to G or S.

Embodiment 5 provides the construct of any one of Embodiments 3-4, wherein each one of C6 and C9 with respect to SEQ ID NO:4 is independently mutated to G or S.

Embodiment 6 provides the construct of any one of Embodiments 3-5, comprising at least one of the following mutations with respect to SEQ ID NO:4: M32Y, S34T, T36E.

Embodiment 7 provides the construct of any one of Embodiments 3-6, comprising each one of the following mutations with respect to SEQ ID NO:4: M32Y, S34T, T36E.

Embodiment 8 provides the construct of any one of Embodiments 1-7, wherein the LINKER is a chemical bond or absent.

Embodiment 9 provides the construct of any one of Embodiments 1-7, wherein the LINKER is a polypeptide comprising 1 100, 1-90, 1-80, 1-70, 1-60, 1-50, 1-40, 1-30, 1-20, 1-10, and/or 1-5 amino acids.

Embodiment 10 provides the construct of any one of Embodiments 1-7 and 9, wherein the LINKER comprises GS and/or GSC.

Embodiment 11 provides the construct of any one of Embodiments 1-7 and 9-10, wherein the LINKER comprises GGGGSGGGGS (SEQ ID NO:5), SSTMVRS (SEQ ID NO:40), and/or SSTMVGS (SEQ ID NO:41).

Embodiment 12 provides the construct of any one of Embodiments 1-7 and 9-10, wherein the LINKER comprises ELKTPLGDTTHTXPRZPAPELLGGP (SEQ ID NO:6), wherein each occurrence of X is C, G, or S, and wherein each occurrence of Z is C, G, or S.

Embodiment 13 provides the construct of any one of Embodiments 1-12, wherein X1 is a covalent bond.

Embodiment 14 provides the construct of any one of Embodiments 1-12, wherein X1 is the peptide of amino acid sequence RAFTNNRKSVSLKKRKKGNRS (SEQ ID NO:3) or a fragment thereof.

Embodiment 15 provides the construct of any one of Embodiments 1-14, wherein X2 is a covalent bond.

Embodiment 16 provides the construct of any one of Embodiments 1-14, wherein X2 is the peptide of amino acid sequence RAFTNNRKSVSLKKRKKGNRS (SEQ ID NO:3) or a fragment thereof.

Embodiment 17 provides the construct of any one of Embodiments 1 and 3-16, wherein the DNAse1 lacks at least a portion of residues 1-22 corresponding to SEQ ID NO: 1.

Embodiment 18 provides the construct of any one of Embodiments 1 and 3-17, wherein the DNAse1 lacks residues 1-22 corresponding to SEQ ID NO:1.

Embodiment 19 provides the construct of any one of Embodiments 1 and 3-18, wherein the DNAse1 comprises at least one of the following mutations with respect to SEQ ID NO:1: Q31R, E35R, Y46H, Y46S, V88N, N96K, D109N, V111T, A136F, R148S, E149N, M186I, L208P, D220N, D250N, A252T, G262N, D265N, and L267T.

Embodiment 20 provides the construct of any one of Embodiments 1 and 3-19, wherein the Fc comprises at least one of the following mutations with respect to SEQ ID NO:4: C6G, C6S, C9G, C9S, M32Y, S34T, and T36E.

Embodiment 21 provides the construct of any one of Embodiments 1 and 3-20, which is selected from the group consisting of SEQ ID NOs:7-17 and 32-35.

Embodiment 22 provides the construct of any one of Embodiments 2-16, wherein the DNAse1L3 lacks at least one of the following: residues 291-305 of SEQ ID NO:2; residues 292-304 of SEQ ID NO:2; residues 296-304 of SEQ ID NO:2; residues A-B of SEQ ID NO:2, wherein A ranges from 291 to 296 and B ranges from 304 to 305.

Embodiment 23 provides the construct of any one of Embodiments 2-16 and 22, wherein the DNAse1L3 comprises at least one of the following mutations with respect to SEQ ID NO:2: E33R, M42T, V44H, V88T, N96K, A127N, V129T, K147S, D148N, L207P, D219N, and V254T.

Embodiment 24 provides the construct of any one of Embodiments 2-16 and 22-23, wherein the Fc comprises at least one of the following mutations with respect to SEQ ID NO:4: C6G, C6S, C9G, C9S, M32Y, S34T, and T36E.

Embodiment 25 provides the construct of any one of Embodiments 2-16 and 22-24, which is selected from the group consisting of SEQ ID NOs:18-28 and 36-39.

Embodiment 26 provides the construct of any one of Embodiments 1-25, which is expressed in a mammalian cell.

Embodiment 27 provides the construct of Embodiment 26, wherein the mammalian cell is stably transfected with human ST6 beta-galatosamide alpha-2,6-sialyltransferase (also known as ST6GAL1).

Embodiment 28 provides the construct of Embodiment 26, wherein the mammalian cell is grown in a cell culture supplemented with sialic acid and/or N-acetylmannosamine (also known as 1,3,4-O-Bu3ManN Ac).

Embodiment 29 provides the construct of any one of Embodiments 1-28, which is soluble.

Embodiment 30 provides a homodimeric construct comprising two independently selected constructs of any one of claims 1, 3-20, and 26-29.

Embodiment 31 provides a homodimeric construct comprising two independently selected constructs of any one of claims 2-16 and 22-29.

Embodiment 32 provides a heterodimeric construct comprising a construct of any one of claims 1, 3-20, and 26-29 and a construct of any one of claims 2-16 and 22-29.

Embodiment 33 provides a method of treating, ameliorating, and/or preventing forms of lupus associated with DNAse1 and/or DNAse1L3 deficiency in a subject, the method comprising administering to the subject a therapeutically effective amount of a construct of any one of Embodiments 1-29.

Embodiment 34 provides the method of Embodiment 33, wherein the lupus comprises systemic lupus erythematosus (SLE).

Embodiment 35 provides a method of treating, ameliorating, and/or preventing diseases and/or disorders associated with inefficient NET hydrolysis (NETolysis) in a subject, the method comprising administering to the subject a therapeutically effective amount of a construct of any one of Embodiments 1-29.

Embodiment 36 provides a method of treating, ameliorating, and/or preventing an autoimmune disorder associated with DNAse1 and/or DNAse1L3 deficiency in a subject, the method comprising administering to the subject a therapeutically effective amount of a construct of any one of Embodiments 1-29.

Embodiment 37 provides the method of Embodiment 36, wherein the autoimmune disorder comprise lupus, thyroid autoimmune disease, and/or Hypocomplementeric Urticarial Vasculitis Syndrome (HUVS).

Embodiment 38 provides a method of treating, ameliorating, and/or preventing pathologic thrombosis in a subject, the method comprising administering to the subject a therapeutically effective amount of a construct of any one of Embodiments 1-29.

Embodiment 39 provides the method of Embodiment 38, wherein the pathologic thrombosis comprises microvascular thrombosis, venous thrombosis, and/or arterial thrombosis.

Embodiment 40 provides the method of any one of Embodiments 38-39, wherein the pathologic thrombosis leads to stroke or makes the subject susceptible to stroke.

Embodiment 41 provides the method of any one of Embodiments 38-40, wherein the pathologic thrombosis comprises neutrophilic thrombosis.

Embodiment 42 provides the method of Embodiment 41, wherein the neutrophilic thrombosis comprises at least one of Anti-Neutrophilic Cytoplasmic Autoantibodies (ANCA) vasculitis, Thrombotic thrombocytopenic purpura (TTP), and Bechet's (or Behcet's) disease or syndrome.

Embodiment 43 provides a method of treating, ameliorating, and/or preventing a myocardial infarction in a subject, the method comprising administering to the subject a therapeutically effective amount of a construct of any one of Embodiments 1-29.

Embodiment 44 provides a method of treating, ameliorating, and/or preventing cancer metastasis in a subject, the method comprising administering to the subject a therapeutically effective amount of a construct of any one of Embodiments 1-29.

Embodiment 45 provides the method of any one of Embodiments 33-44, wherein in the DNAse1 and/or DNAse1L3 precursor construct the signal peptide sequence is conjugated to the N-terminus of the DNAse1 and/or DNAse1 L3 polypeptide.

Embodiment 46 provides the method of any one of Embodiments 33-45, wherein the construct is a secreted product of a DNAse1 and/or DNAse1L3 precursor construct expressed in a mammalian cell, wherein the DNAse1 and/or DNAse1L3 precursor construct comprises a signal peptide sequence and a DNAse1 and/or DNAse1L3 polypeptide, wherein the DNAse1 and/or DNAse1L3 precursor construct undergoes proteolytic processing to yield the DNAse1 and/or DNAse1L3 construct.

Embodiment 47 provides the method of Embodiment 46, wherein the mammalian cell is stably transfected with human ST6 beta-galatosamide alpha-2,6-sialyltransferase (also known as ST6GAL1).

Embodiment 48 provides the method of Embodiment 46, wherein the mammalian cell is grown in a cell culture supplemented with sialic acid and/or N-acetylmannosamine (also known as 1,3,4-O-Bu3ManNAc).

Embodiment 49 provides the method of any one of Embodiments 33-48, wherein the construct is administered acutely or chronically to the subject.

Embodiment 50 provides the method of any one of Embodiments 33-48, wherein the construct is administered locally, regionally, parenterally, and/or systemically to the subject.

Embodiment 51 provides the method of any one of Embodiments 33-50, wherein the construct, and/or its precursor construct, is delivered on an encoded vector to the subject, wherein the vector encodes the construct or precursor construct, which is transcribed and translated from the vector upon administration of the vector to the subject.

Embodiment 52 provides the method of any one of Embodiments 33-51, wherein the construct is administered to the subject by at least one route selected from the group consisting of subcutaneous, oral, aerosol, inhalational, rectal, vaginal, transdermal, subcutaneous, intranasal, buccal, sublingual, parenteral, intrathecal, intragastrical, ophthalmic, pulmonary, and topical.

Embodiment 53 provides the method of any one of Embodiments 33-52, wherein the construct is administered to the subject as a pharmaceutical composition further comprising at least one pharmaceutically acceptable carrier.

Embodiment 54 provides the method of any one of Embodiments 33-53, wherein the construct comprises a homodimeric construct comprising two independently selected constructs of any one of Embodiments 1, 3-20, and 26-29; a homodimeric construct comprising two independently selected constructs of any one of Embodiments 2-16 and 22-29; and/or a heterodimeric construct comprising a construct of any one of Embodiments 1, 3-20, and 26-29 and a construct of any one of Embodiments 2-16 and 22-29.

Embodiment 55 provides the method of any one of Embodiments 33-54, wherein the subject is a mammal.

Embodiment 56 provides the method of Embodiment 55, wherein the mammal is human.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

What is claimed:

1. A construct, wherein the construct comprises a polypeptide comprising:
a DNAse domain, wherein the DNAse domain comprises a DNAse1 domain or a DNAse1L3 domain; and
an Fc domain, wherein the Fc domain comprises one or more amino acid residue modifications for enhanced endosomal recycling relative to a human immunoglobulin Fc domain.

2. The construct of claim 1, wherein the Fc domain is C-terminal to the DNAse domain.

3. The construct of claim 1, wherein the one or more amino acid residue modifications further provide for reduced Fc dimerization relative to a human immunoglobulin Fc domain.

4. The construct of claim 1, wherein the Fc domain is modified from a human immunoglobulin 1 (IgG1), human immunoglobulin 2 (IgG2), human immunoglobulin 3 (IgG3), or human immunoglobulin 4 (IgG4).

5. The construct of claim 1, wherein the Fc domain is modified from a human immunoglobulin 1 (IgG1).

6. The construct of claim 5, wherein at least one of C6 and C9 with respect to SEQ ID NO:4 is independently modified to G or S.

7. The construct of claim 5, wherein C6 and C9 with respect to SEQ ID NO:4 are independently modified to G or S.

8. The construct of claim 5, wherein the Fc domain comprises at least one of the following modifications with respect to SEQ ID NO:4; C6G, C6S, C9G, C9S, M32Y, S34T, and T36E.

9. The construct of claim 5, wherein the Fc domain comprises at least one of the following modifications with respect to SEQ ID NO:4 M32Y, S34T, and T36E.

10. The construct of claim 9, wherein the construct further comprises a linker between the DNase domain and the Fc domain.

11. The construct of claim 10, wherein the linker is a polypeptide comprising 1-100, 1-90, 1-80, 1-70, 1-60, 1-50, 1-40, 1-30, 1-20, 1-10, or 1-5 amino acids.

12. The construct of claim 1, wherein the construct further comprises a linker between the DNase domain and the Fc domain.

13. The construct of claim 12, wherein the linker is a polypeptide comprising 1-100, 1-90, 1-80, 1-70, 1-60, 1-50, 1-40, 1-30, 1-20, 1-10, or 1-5 amino acids.

14. The construct of claim 1, wherein the construct is produced by a Chinese Hamster Ovary (CHO) cell line that had been stably transfected with human ST6 beta-galactosamide alpha-2,6-sialyltransferase (ST6GAL1).

15. The construct of claim 14, wherein the CHO cell line is supplemented with sialic acid or N-acetylmannosamine (1,3,4-O-Bu3ManNAc).

16. The construct of claim 1, wherein the DNAse domain is a human DNase1 domain and comprises one or more modifications relative to amino acid residues according to human DNAse1 protein of SEQ ID NO:1.

17. The construct of claim 16, wherein the human DNAse1 domain lacks residues 1-22 corresponding to SEQ ID NO:1.

18. The construct of claim 16, wherein the human DNAse1 domain comprises at least one of the following modifications with respect to SEQ ID NO:1: Q31R, E35R, Y46H, Y46S, V88N, N96K, D109N, V111T, A136F, R148S, E149N, M186I, L208P, D220N, D250N, A252T, G262N, D265N, and L267T.

19. The construct of claim 1, wherein the DNAse domain is a human DNase1L3 domain and comprises one or more modifications relative to amino acid residues according to human DNAse1L3 protein of SEQ ID NO:2.

20. The construct of claim 19, wherein the human DNase1L3 domain lacks residues 1-20 corresponding to SEQ ID NO:2.


SEQUENCE LISTING (43 sequences)

SEQ ID NO: 1 - protein, 282 aa, Homo sapiens [sequence omitted]

SEQ ID NO: 2 - protein, 305 aa, Homo sapiens
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 3 - protein, 21 aa, synthetic construct
  source 1..21: note = Chemically synthesized
RAFTNNRKSVSLKKRKKGNRS

SEQ ID NO: 4 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 10 aa, synthetic construct
  source 1..10: note = Chemically synthesized
GGGGSGGGGS

SEQ ID NO: 6 - protein, 25 aa, synthetic construct
  VAR_SEQ 13: note = X = Cys, Gly, or Ser
  VAR_SEQ 16: note = X = Cys, Gly, or Ser
  source 1..25: note = Chemically synthesized
ELKTPLGDTTHTXPRXPAPELLGGP

SEQ ID NO: 7 - protein, 511 aa, synthetic construct
  source 1..511: note = Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 511 aa, synthetic construct
  source 1..511: note = Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 511 aa, synthetic construct
  source 1..511: note = Chemcially synhesized
MRGMKLLGALLALAALLQGAVSLKIAAFNIQTFGRTKMSNATLVSYIVQILSRYDIALVQEVRDSHLTAVGKLLDNLNQDAPDTYHYNVSEPLGRNSYKERYLFVYRPNQTSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYNLSNQTAQAISDHYPVEVMLKGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 553 aa, synthetic construct
  source 1..553: note = Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKRAFTNNRKSVSLKKRKKGNRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 11 - protein, 532 aa, synthetic construct
  source 1..532: note = Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 12 - protein, 532 aa, synthetic construct
  source 1..532: note = Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKRAFTNNRKSVSLKKRKKGNRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 519 aa, synthetic construct
  source 1..519: note = Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGGGGSGGGGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 534 aa, synthetic construct
  VAR_SEQ 295: note = X = Cys, Gly, or Ser
  VAR_SEQ 298: note = X = Cys, Gly, or Ser
  source 1..534: note = Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKELKTPLGDTTHTXPRXPAPELLGGPDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 512 aa, synthetic construct
  source 1..512: note = Chemically synthesized
MRGMKLLGALLALAALLQGAVSLKIAAFNIRTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLQGAVVPDSALPFNFQAAYGLSDQLAQAISDHYPVEVMLKGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 512 aa, synthetic construct
  source 1..512: note = Chemically synthesized
MRGMKLLGALLALAALLQGAVSLKIAAFNIRTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLQGAVVPDSALPFNFQAAYGLSNQTAQAISDHYPVEVMLKGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 512 aa, synthetic construct
  source 1..512: note = Chemically synthesized
MRGMKLLGALLALAALLQGAVSLKIAAFNIRTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLQGAVVPNSTLPFNFQAAYGLSDQLAQAISDHYPVEVMLKGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 534 aa, synthetic construct
  source 1..534: note = Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 534 aa, synthetic construct
  source 1..534: note = Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 555 aa, synthetic construct
  source 1..555: note = Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 21 - protein, 576 aa, synthetic construct
  source 1..576: note = Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSRAFTNNRKSVSLKKRKKGNRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 22 - protein, 555 aa, synthetic construct
  source 1..555: note = Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSRAFTNNRKSVSLKKRKKGNRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 542 aa, synthetic construct
  source 1..542: note = Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGGGGSGGGGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 557 aa, synthetic construct
  VAR_SEQ 318: note = X = Cys, Gly, or Ser
  VAR_SEQ 321: note = X = Cys, Gly, or Ser
  VAR_SEQ 336: note = X = Cys, Gly, or Ser
  source 1..557: note = Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSELKTPLGDTTHTXPRXPAPEFLGGPDKTHTXPPZPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 519 aa, synthetic construct
  VAR_SEQ 298: note = X = Cys, Gly, or Ser
  VAR_SEQ 301: note = X = Cys, Gly, or Ser
  source 1..519: note = Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSGSDKTHTXPPZPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 535 aa, synthetic construct
  source 1..535: note = Chemically synthesized
MSRELAPLLLLLLSIHSALAMRICSFNVRSFGRSKQEDKNATDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 535 aa, synthetic construct
  source 1..535: note = Chemically synthesized
MSRELAPLLLLLLSIHSALAMRICSFNVRSFGRSKQEDKNATDVIVKVIKRCDIILVMEIKDSNNRICPILMEKLNRNSRRGITYNYVISSRLGRKTYKEQYAFLYKEKLVSVKRSYHYHDYQDGDNDTFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 535 aa, synthetic construct
  source 1..535: note = Chemically synthesized
MSRELAPLLLLLLSIHSALAMRICSFNVRSFGRSKQEDKNATDVIVKVIKRCDIILVMEIKDSNNRICPILMEKLNRNSRRGITYNYTISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 283 aa, Mus musculus
MRYTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCECGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKI

SEQ ID NO: 30 - protein, 310 aa, Mus musculus
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRS

SEQ ID NO: 31 - protein, 222 aa, Mus musculus
GCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 32 - protein, 512 aa, synthetic construct
  source 1..512: note = Chemically synthesized
MRYTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCECGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKISSTMVRSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 33 - protein, 513 aa, synthetic construct
  source 1..513: note = Chemically synthesized
MRYTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPFIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKISSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 513 aa, synthetic construct
  source 1..513: note = Chemically synthesized
MRYTGLMGTLLTLVNLLQLAGTLRIAAFNIRTFGRTKMSNATLSVYFVKILSRYDIAVIQEVRDSHLVAVGKLLDELNRDKPDTYRYNVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKISSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 35 - protein, 513 aa, synthetic construct
  source 1..513: note = Chemically synthesized
MRYTGLMGTLLTLVNLLQLAGTLRIAAFNIRTFGRTKMSNATLSVYFVKILSRYDIAVIQEVRDSHLVAVGKLLDELNRDKPDTYRYNVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQTAEAISDHYPVEVTLRKISSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 36 - protein, 539 aa, synthetic construct
  source 1..539: note = Chemically synthesized
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRSSSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 37 - protein, 519 aa, synthetic construct
  source 1..519: note = Chemically synthesized
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 38 - protein, 539 aa, synthetic construct
  source 1..539: note = Chemically synthesized
MSLHPASPRLASLLLFILALHDTLALRLCSFNVRSFGRSKKENHEAMDIIVKIIKRCDLILLMEIKDSSNNICPMLMEKLNGNSRRSTTYNYVISSRLGRKTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRSSSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 39 - protein, 539 aa, synthetic construct
  source 1..539: note = Chemically synthesized
MSLHPASPRLASLLLFILALHDTLALRLCSFNVRSFGRSKKENHEAMDIIVKIIKRCDLILLMEIKDSSNNICPMLMEKLNGNSRRSTTYNYVISSRLGRKTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSNGTFDFQKAYDLSEEEALDVSDHFPVEFKLQSSRAFTNNRKSVSLKKRKKGNRSSSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 40 - protein, 7 aa, synthetic construct
  source 1..7: note = Chemically synthesized
SSTMVRS

SEQ ID NO: 41 - protein, 7 aa, synthetic construct
  source 1..7: note = Chemically synthesized
SSTMVGS

SEQ ID NO: 42 - protein, 507 aa, synthetic construct
  source 1..507: note = Chemically synthesized
MGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKISSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 43 - protein, 530 aa, synthetic construct
  source 1..530: note = Chemically synthesized
LASLLLFILALHDTLALRLCSFNVRSFGRSKKENHEAMDIIVKIIKRCDLILLMEIKDSSNNICPMLMEKLNGNSRRSTTYNYVISSRLGRKTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRSSSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK